(19) United States
(12) Patent Application Publication
(10) Pub. No.: US 2006/0148080 A1
(43) Pub. Date: Jul. 6, 2006

(54) METHODS FOR SUPPORTING AND PRODUCING HUMAN CELLS AND TISSUES IN NON-HUMAN MAMMAL HOSTS

(76) Inventor: Paul Diamond, Secaucus, NJ (US)

Correspondence Address: PAUL DIAMOND, 942 SCHOPMANN DRIVE, NO. 2, SECAUCUS, NJ, 07094, US

(21) Appl. No.: 11/162,715

(22) Filed: Sep. 20, 2005

Related U.S. Application Data
(60) Provisional application No. 60/640,445, filed on Dec. 30, 2004.

(51) Int. Cl. C12N 5/08

(52) U.S. Cl. 435/366

(57) ABSTRACT

Provided are methods for culturing and producing human cell containing compositions, such as organs and blood, in non-human mammal hosts and for the preparation of animal-hosted human cell containing compositions for transplantation to human subjects. In one aspect, non-human hosts genetically modified or treated to reduce the expression of xenoantigens, such as alpha-galactosyl epitopes, are used to reduce the transfer of xenoantigens to the hosted human cells. In another aspect, non-human hosts genetically modified to express or over-express molecules that promote immunological tolerance to a graft, such as hDAF, are used so that the molecules are transferred to the hosted human cells. These aspects facilitate the immunological acceptance of the human cell containing compositions upon transplantation to a human subject. Still another aspect provides conditioning treatments that facilitate the immunological acceptance of human cell containing compositions that have been supported in non-human hosts upon their transplantation to a human subject.

### CROSS-REFERENCE TO RELATED APPLICATIONS

Priority is claimed to U.S. provisional application Ser. No. 60/640,445 filed Dec. 30, 2004, which is hereby incorporated by reference in its entirety.

### FIELD OF THE INVENTION

The invention relates to the fields of transplant biology and xenotransplantation.

### BACKGROUND

There is an ongoing need for human organs, tissues and cells for transplantation to human patients in need of such transplants. This shortage is due, at least in part, to each of the following reasons: a limited number of available, immunologically suitable donor organs and tissues; the limited period of time for which a donor material is suitable for transplant to a patient after it has been explanted from the donor; and the logistical and economic difficulties associated with delivering suitable donor material, especially solid organs to transplant locations.

There has been ongoing research in the art to address these problems. For example, the field of xenotransplantion aims to provide animal organs or chimeric human-animal organs suitable for transplantation to human patients. A key challenge in this field relates to preventing the immunological rejection of non-human animal cells by the human immune system following transplantation. On another front, efforts are being made to develop improved organ preservation solutions to prolong the useful life of donor organs and tissues. Still other work is directed to developing improved devices for the extracorporeal support of living donor organs. These devices are similar to heart-lung machines in that they perfuse the subject organ with a medium providing oxygen and nutrients. One such device is the Transmedics Portable Organ Preservation System (POPS). Despite these promising technologies, the need for donor organs and tissues for human patients remains unmet.

### SUMMARY

The invention provides improved methods for culturing and producing human cell containing compositions in non-human mammal hosts and for the preparation of animal-hosted human cell containing compositions for transplantation to human subjects.

In one aspect, non-human hosts genetically modified or otherwise treated to reduce the expression of xenoantigens, such as alpha-galactosyl epitopes, are provided in order to reduce the transfer of xenoantigens to the hosted human cells. In another aspect, non-human hosts genetically modified to express or over-express molecules that promote immunological tolerance to a graft, such as hDAF (CD55) and/or MIRL (CD59), are provided so that the molecules are transferred to the hosted human cells. These aspects facilitate the immunological acceptance of the human cell containing compositions upon transplantation to a human subject. Still another aspect provides conditioning treatments that facilitate the immunological acceptance of human cell containing compositions that have been supported in non-human mammal hosts, upon their transplantation to a human being.

One aspect of the invention provides a method of supporting human cells in a living state that includes the step of: supporting human cells in a living state in a non-human, mammal host, wherein the host is modified or treated to reduce the expression of at least one xenoantigen defined with respect to a normal human immune system.

A related aspect of the invention provides a method for supporting human cells in a non-human, mammalian host animal, that includes the steps of: transplanting human cells to a non-human, mammal host, wherein the host is at least substantially immunologically tolerant of the transplanted human cells, wherein the human cells are supported in a living state by the host, and wherein the host is modified or treated to reduce the expression of at least one xenoantigen defined with respect to a normal human immune system.

The methods may further include a step of treating the host to reduce the expression of the at least one xenoantigen. The methods may further include a step of providing the host, wherein the provided host includes at least one modification that reduces the expression of the at least one xenoantigen. The methods may further include a step of selectively killing host cells of a host organ or tissue to promote population of the organ or tissue with human cells. The methods may further include a step of selectively killing host cells of a composition that comprises host cells and currently or previously hosted human cells in order to provide a composition that is at least substantially entirely cellularly human.

In one variation of the methods, the host is at a fetal or neonatal stage at the time the human cells are transplanted and at least some of the human cells incorporate into one or more organs and/or tissues of the host. In another variation, the host is at a post-birth stage of development, for example, at a sexually mature stage. In another variation of the methods, at least some of the human cells introduced into the host are capable of directly or indirectly giving rise to human hematopoietic cells and human hematopoietic cells are produced within the host. The human hematopoieitic stem cells produced within the host may be collected from the host.

A further aspect of the invention provides a non-human animal, that includes: a non-human mammal host; and human cells supported in a living state by the host, wherein, the host is at least substantially immunologically tolerant of the human ells, and wherein, the host has reduced expression of at least one xenoantigen defined with respect to a normal human immune system.

A related aspect of the invention provides a non-human animal, that includes: a non-human mammal host; and human cells supported in a living state by the host, wherein, the host is at least substantially immunologically tolerant of the human cells, and wherein, the host (i) includes at least one modification, such as a genetic modification, that reduces the expression of at least one xenoantigen defined with respect to a normal human immune system. or (ii) has reduced expression of at least one xenoantigen defined with respect to a normal human immune system as a result of treatment of the host or (iii) both (i) and (ii).

The hosts may further include at least one genetic modification rendering at least some of the native host cells selectively and conditionally killable. This effect may be at least substantially general to the host cells or it may be at least substantially restricted to one or more host organ or tissues.

In one variation of the methods or animals, at least some of the human cells are human hematopoietic stem cells and/or human hematopoietic progenitor cells. In another variation of the methods or animals, at least some of the human cells produce human hematopoietic cells, such as differentiated human hematopoietic cells, within the host. In still another variation of the methods or animals, the hematopoietic system of the host is at least partially populated by human cells and produces at least some human hematopoietic cells, such as erythrocytes, platelets, lymphocytes, for example, B and T cells, and dendritic cells.

In connection with the methods and animals, the at least one xenoantigen may include alpha-galactosyl epitopes. The at least one xenoantigen may more specifically include alpha(1,3)galactosyltransferase (GGTA1)-synthesized alpha-galactosyl epitopes and/or iGb3 synthase-synthesized alpha-galactosyl epitopes. A host may for example, include one or more genetic modifications that inactivate at least one or each of the alleles of the GGTA1 gene. The at least one xenoantigen may comprise NeuGC epitopes, such as CMP-NeuAc hydroxylase-synthesized NeuGC epitopes. A host may, for example, include one or more genetic modifications that inactivate at least one or each of the alleles of the CMP-NeuAc hydroxylase gene, in order to reduce the expression of NeuGC epitopes.

Another aspect of the invention provides a method for maintaining a human organ or human tissue for a period of time in a non-human, mammal host that includes the step of: transplanting at least part of a functionally developed or still developing human organ, tissue or body part to a non-human, mammal host, wherein the host animal is at least substantially immunologically tolerant of the transplanted organ, tissue or body part, and wherein the transplanted human organ, tissue or body part is supported in a living state by the host. The method may comprise a further step of selectively killing native host cells that may be present in the organ, tissue or body part, for example, concurrent with and/or after isolating the human organ, tissue or body part from the host after a period of support therein. The host may, for example, include at least one genetic modification that permits at least some or at least substantially all of the native host cells to be selectively and conditionally killed, while the human cells remain at least substantially unharmed. The genetic modification may, for example, comprise a transgene permitting inducible or constitutive expression of a suicide gene. The host may, for example, include at least one genetic modification that causes or increases the expression by the host of at least one tolerance-promoting biomolecule. The method may include a step of supporting the organ, tissue or body part on an extracorporeal support device for a period of time following removal from the host.

A related aspect of the invention provides a non-human animal that includes a non-human mammal host supporting a human transplant in a living state, the transplant comprising at least part of a functionally developed or still developing human organ, tissue or body part to a non-human, mammal host, wherein the host is at least substantially immunologically tolerant of the transplanted organ, tissue or body part, and wherein the host includes at least one genetic modification that permits at least some or at least substantially all of the native host cells to be selectively and conditionally killed, while the human cells remain at least substantially unharmed. The host may be at a fetal or post-birth stage of development.

Another related aspect of the invention provides a non-human animal that includes a non-human mammal host supporting a human transplant in a living state, the transplant comprising at least part of a functionally developed or still developing human organ, tissue or body part to a non-human, mammal host, wherein the host is at least substantially immunologically tolerant of the transplanted organ, tissue or body part, and wherein the host includes at least one genetic modification that causes or increases the expression by the host of at least one tolerance-promoting biomolecule. The host may be at a fetal or post-birth stage of development

In any of the methods and animals, the hosts may also be provided with expression or increased expression of at least one tolerance-promoting biomolecule, such as a GPI-anchored form of a tolerance promoting biomolecule. For example, a non-human mammal host comprising a transgene that drives the expression of a tolerance promoting molecule such as a human complement inhibitor, may be used.

In any of the methods, the human cells may be supported in a living state by the animal host for any period of time, such as but not limited to, at least 1, 2, 3, 4, 5, 7, 14, 30, 90 or 180 days. Any of the methods may also comprise a further step of actively maintaining the transplant in the animal host. Actively maintaining the transplant in the animal host may, for example, include husbandry of the host animals, feeding the host animals, providing habitation for the host animal and/or caring for the host animal during the period in which the human transplant is present in and supported by the host animal. Any of the methods may also include a further step in which at least some of the human cells, in whatever form, may be transplanted to a human subject from the animal host after the period of support in the host.

### BRIEF DESCRIPTION OF THE DRAWINGS

FIGS. 1A, 1B and 1C are schematic representations of transgene constructs that can be used to produce transgenic animals expressing suicide genes.

### DETAILED DESCRIPTION

The invention provides improved methods for culturing and producing human cells in non-human mammal hosts and for preparing human cell containing compositions that have been supported in such hosts for transplantation to human subjects. In one aspect, the invention provides for the use of animal hosts with reduced transferable xenoantigen expression in order to reduce the transfer of one or more xenoantigens from the host to the hosted cells.

The human cells transplanted to and supported by the host may be in any form. In one embodiment, human organs or tissues, or parts thereof, from living or deceased human donors are supported in a non-human mammal host. Complex composite tissues or sections thereof, such as parts of a body, may also be supported in/by a non-human mammal host according to the invention. The human organs or tissues may be solid organs or tissues or dispersed organs or tissues (e.g., blood, blood marrow). Some solid tissues may also include at least part of a dispersed tissue, such a bone containing blood marrow. In one variation, the human organ(s) or tissue(s) is, at the time of transplantation to the non-human host, already functionally developed and may, for example, be from a donor at a post-birth stage of development. In a different variation, the human organ(s) or tissue(s) are, at the time of transplantation to the non-human host still be in an anlagen stage of development. Such anlagen may, upon transplantation to the host, be permitted to continue their growth and differentiation into a functioning organ or tissue. Methods for transplanting anlagen to a host mammal for development are, for example, disclosed in U.S. Pub. Nos. 20040191228, 20040136972, 20040082064, 20030198628, 20030096016 and 20030086909, each of which is incorporated by reference herein in its entirety.

Functionally developed solid human donor organs and tissues that can be supported by non-human mammal hosts according to the invention include, but are not limited to: heart, lung, kidney, liver, pancreas, gall bladder; spleen, urinary bladder; glands; trachea; esophagus; stomach; small intestine; large intestine; muscles; bone; cartilage, vascular tissue (e.g., arteries, veins); lymphatic tissue (e.g., ducts, nodes); reproductive tissue structures; neural tissue, nerve tissue, skin (hair-bearing and hairless); soft solid tissues; hard solid tissues; complex composite structures including, but not limited to joints and appendages (e.g., arm, hand, finger), reproductive organs; and substantial parts or sections thereof. In one embodiment, a brain or a substantial part thereof is expressly excluded from the human organs and tissues that may be supported in a non-human host according to the various aspects of the invention. A functionally developed human organ or tissue can be obtained in a medically and ethically appropriate manner from any stage of human donor, for example, from an adult, adolescent or child. Thus, functionally-developed human organs and tissues that have not yet reached their full adult size, i.e., are still growing in size, are also within the scope of the invention. Further, the donor may be a living donor or a deceased donor. For example, a lobe of the liver, a kidney, a lung or a section of skin could all be provided by a living donor. Anlagen that can be supported by non-human mammal hosts according to the invention include but are not limited to pancreatic anlagen, kidney anlagen and lung anlagen.

In another embodiment, the human cells supported in the animal host form part of one or more chimeric organs or tissues that include the human cells and (i.) cells of the host and/or (ii.) cells of another non-human mammal. In one method, human cells capable of engrafting in or giving rise to part(s) of an organ or tissue of the host are introduced into the host. In one variation of this method, the human cells are directly or indirectly introduced into an organ or tissue of a host that is at a post-birth stage of development. In another variation of the method, human cells are introduced into an organ or tissue of a first non-human mammal that may or may not be genetically modified or otherwise modified or treated to reduced xenoantigen expression, to form one or more chimeric organs or tissues and then at least part of one of the chimeric organs or tissues is transplanted to a second non-human mammal genetically modified or otherwise modified or treated to reduced xenoantigen expression.

In another method, human cells capable of engrafting in or giving rise to part(s) of an organ or tissue of the host are introduced into the host during its fetal phase. Introduction of cells into the host during its fetal stage can advantageously result in the codevelopment of the human cells in and with the host organs and/or tissue to a significant extent. Introduction of human cells into a fetal, non-human mammal, such as a pig or sheep to form chimeric organs and tissue is, for example, disclosed in U.S. Pub. Nos. 20020100065 (application Ser. No. 09/895,895) and 20030096410 (application Ser. No. (09/178,036), and WO 2004/027029 A2 and its corresponding U.S. national phase, application Ser. No. 10/527,587, each of which is incorporated by reference herein in its entirety.

In still another embodiment, cells that are capable of producing hematopoietic cells (blood cells) are introduced into and supported in the non-human mammal host that is genetically modified or otherwise modified or treated to reduce the expression of one or more xenoantigens. In this manner, a non-human mammal may serve as a living bioreactor to produce one or more human hematopoietic cell types of interest, for example, one or more of the types of white blood cells and/or erythrocytes. Advantageously, the use of hosts having reduced expression of or more xenoantigens, according to one aspect of invention, reduces the transfer of xenoantigens to the host human cells and thereby provides product human cells that are less xenoantigenic upon transplantation to a human subject. For example, since erythrocytes receive GPI-linked xenoantigens from various sources and deposit GPI-linked xenoantigens on endothelium, a host genetically modified or otherwise modified or treated to reduce the level of expression of one or more xenoantigens, such as reduced or eliminated GGTA1-mediated production of alpha-galactosyl epitopes, can be used for producing human erythrocytes. At least several different cell types and strategies can be used to introduce human cells that give rise to human hematopoietic cells into a non-human mammal host in order to produce human hematopoietic cells in the host. A transplant of adult or fetal nucleated bone marrow cells can be engrafted to a fetal or post-birth stage non-human mammal host. In performing a human bone marrow to post-birth-stage animal host transplant, the host marrow may be prepared by chemical and/or radiological myeloabalation to facilitate the engraftment of the human marrow cells. Such myeloablation may not be necessary when the animal host recipient is a fetus. Human bone marrow, human cord blood and human G-CSF mobilized peripheral blood cells may also be introduced into an immunologically tolerant, non-human animal fetus such as a pig, sheep or rodent (mouse, rat, guinea pig, capybara, etc.) to give rise durable, multi-lineage human hematopoiesis in the host. The human cells may be introduced by any method such as intraperitoneal injection or intravascular infusion. Cell fractions enriched for human hematopoietic stem cells (HSC) may also be obtained from bone marrow, cord blood or mobilized peripheral blood cells and introduced into fetal hosts for engraftment and human hematopoiesis. For example, human cell fractions enriched for CD34⁺ Lin⁻ phenotypes may be used. In still another method, human hematopoietic activity can be transferred to a xenoantigen-reduced, non-human mammal host by transplanting human red marrow-containing bone to the host where it is supported in a living state.

It should be understood that the term “introduced cells” or “transplanted cells” and the like, when used herein to describe human cells presently hosted in a non-human mammal host or that were supported in such a host, can encompass not only the cells in the state in which they were originally introduced into the non-human mammal hosts, but also any cells derived from the introduced cells by the processes of cell division and/or differentiation and/or dedifferentiation. For example, some human tissues such as muscle that are introduced into a non-human mammal host according to the invention may remain at least substantially mitotically inactive over a period of time of support within the host. On the other hand, stem cells, such as hematopoietic stems cells, may undergo significant expansion (renewal) and/or differentiation into a variety of further restricted progenitor cell types and/or differentiated cells, such as mature hematopoietic cells, hepatocytes, and liver biliary duct cells. The term “transplanting” and related forms of the term as used herein may include, but are not limited to, transfer of cells or cell-containing compositions from one body to another body. Accordingly, for example, transplanting may also include implanting, injecting or otherwise introducing cells of a cell line into a recipient animal or human body. Further, the terms “transplanting” and “introducing” and related forms of these terms may include, but are not limited to, cases where the transplanted or introduced human cells or human cell containing compositions are enclosed within a recipient. For example, transplanted skin or appendages may be exposed to the outer environment while being attached to and supported by a host, in a manner characteristic of such tissues. In a contrasting example, transplanted or introduced organs such as a liver, pancreas, heart or spleen may be entirely enclosed by the recipient host.

Accordingly, general and specific methods for culturing human cells in a non-human animal, such as a non-human mammal, are provided that include the steps of: introducing human cells in any form into a non-human host animal, such as a non-human mammal, that is at least substantially immunologically tolerant of the human cells and which is genetically modified, or otherwise modified or treated, to reduce the expression of at least one xenoantigen (defined with respect the human immune system), such as alpha-galactosyl epitopes and/or NeuGc epitopes, so that transfer of the xenoantigens from the host to the human cells is reduced; and supporting the cells in a living state in the host animal for a period of time, such as at least 2 days, or at least one week or at least one month. A related method further includes the step of removing at least some of the human cells or a human cell containing composition from the animal host.

In the case that the host is modified to reduce the expression of alpha-galactosyl epitopes, it may for example be modified (such as genetically modified in any prior or current generation) or treated to reduce the expression of alpha(1,3)galactosyltransferase-produced and/or iGb3 synthase-produced alpha-galactosyl epitopes.

In another variation, a composition of a mixture of human cells and host cells is then removed from the host and the host cells are selectively killed to obtain an at least substantially pure composition of human cells (with respect to living cells). A host animal and/or the hosted xenogeneic cells may be genetically modified to enable the selective deletion of the host cells.

In a different variation, a human cell containing composition is then removed from the animal host and is post-conditioned with at least one enzymatic treatment to remove host xenoantigens.

In still another variation, a human cell containing composition that was hosted in the non-human animal is removed from the host and placed on extracorporeal support, for example, for at least 1, 2, 3, 4, 7, or 30 days.

In a further variation of the embodiment or any of its aforementioned variations, a human cell containing composition, for example, a functionally developed organ or tissue, such as a functionally developed, solid human organ or tissue, can then be transplanted to a human patient in need thereof.

Various aspects and examples of the invention are described in the following sections.

Tolerance of Animal Host to Human Cells

The host animal may be at least substantially immunologically tolerant to the transplanted human cell containing composition. The invention is not limited to the manner in which a suitably tolerant host is obtained or provided and any method or combination(s) of methods can be used.

One method provides a host suitably tolerant of human tissue by using a fetal, non-human, mammal host, such as a fetal pig or sheep or rodent (mouse, rat, guinea pig, capybara, etc.), since the fetal mammalian environment is tolerant to foreign human cells and tissue. In this embodiment, the fetal non-human mammal is the host animal, but it should be understood that such a host can, according to the invention, continue to support the introduced human cell-containing composition following birth.

Another method provides a host suitably tolerant of human tissue by providing a post-birth-stage, non-human mammal host, such as a pig, that was contacted during fetal development with human cells and/or human cellular antigens. In one method, tolerance to foreign human cells and tissue is imparted by infusing human bone marrow cells into a fetal non-human mammal where they may engraft. Following birth, such an animal has improved tolerance toward human tissue.

A further method provides a non-human host at a post-birth stage of development that is suitably tolerant of human cells by depleting the immune system of the host using chemical treatment and/or irradiation. Optionally, the ablated bone marrow of the host can be replaced with human bone marrow cells. For example, x-ray or gamma-radiation, sufficient to destroy at least substantially all of the host's bone marrow can be employed. U.S. Pat. No. 6,018,096. Chemical ablation, with or without radiation, of at least substantially all of the bone marrow of the animal host using myeloablative agents such as cyclophosphamide, busulfan or combinations thereof can also be employed to obtain substantial tolerance to human tissue.

Another method provides a non-human mammal host suitably tolerant of human tissue by providing a host conditioned according to the method of U.S. Pat. No. 6,296,846, which is incorporated by reference herein.

Another method provides a host suitably tolerant of human tissue by providing a host that is genetically immunocompromised. Such a host may comprise at least one genetic modification or mutation, intentionally introduced (targeted) or otherwise arising at any time in the past or in any previous generation, that disrupts the host's immune system. For example, non-human mammals homozygous for mutations in the Rag1 gene (recombination activating gene 1) are characterized by a deficiency in both T-cells and B-cells. Rag1 gene-deficient mice, obtained by knockout methods, have been previously described and are well known in the art (Mombaerts, P. et al., RAG-1-deficient mice have no mature B and T lymphocytes Cell, (1992) 68 (5), 869-77, which is incorporated by reference herein in its entirety). Swine genetically modified to be deficient in the Rag1 gene are disclosed in U.S. Pub. No. 20050155094 (application Ser. No. 10/503,464), which is incorporated by reference herein in its entirety. Non-human mammals homozygous for mutations in the Prkdc gene, known in the art as a type of SCID (severe combined immune deficiency), are also characterized by a deficiency in both T-cells and B-cells. Non-human mammals homozygous for mutations in the Foxn1 gene, known in the art as nude mammals, are characterized by thymic dysgenesis with deficiency in T-cells and partial defects in B-cell development. All three of these mutants are also characterized by secondary immune defects relating, for example, to antigen presenting cells (APCs) and natural killer cells (NK cells).

A genetically modified, conditionally immunodeficient non-human mammal can also be used as a suitably tolerant host animal pursuant to conditionally inducing the immunodeficiency. For example transgenic mammals including a transgene construct in which expression of a protoxin-to-toxin converting enzyme type of suicide gene is under control of a lymphocyte specific promoter, such as the jak3 (Janus kinase 3) promoter, can be produced. T-cell and B-cell deficiency is conditionally induced by providing the transgenic animal with the protoxin (prodrug). The production of such a mammal is, for example, further provided by International Pub. No. WO 2004/027029 A2 and its corresponding U.S. national phase, application Ser. No. 10/527,587.

Another manner in which a non-human mammal host at least substantially tolerant of human cells can be provided is by transplanting the human donor organ or tissue to an immunologically privileged site in the host. Reported immune privileged sites include, for example, the testes, the eye (anterior chamber, cornea, and retina), the brain and the placenta. It has also been reported that xenogeneic tissue transplantations under a non-human mammal host's kidney capsule can, at least in some cases, avoid rejection.

Transplantation for Solid Organ and Tissue Embodiments

The surgical techniques for the transplantation of various solid organs and tissues from one individual to another, for example, from one pig to another, from a pig to sheep, and from a pig to a primate are well developed. An organ or tissue (such as a composite or non-composite tissue structure) or part thereof that includes human cells can be transplanted to any suitable location of a non-human mammal host where it can be supported in a living state by the host. Blood supply to vascularized organs and tissues or parts thereof can be established, for example, by anastomosing host and donor arterial vessels to each other and, if required, host and donor venous vessels to each other. Vascular grafts from the donor or host can also be used, if needed, to provide inflow and outflow of blood to the donor organ or tissue in the host. For smaller donor organs such as glands or thinner donor tissues such as skin, a sufficient blood supply can, for example, be established over a short period by placing the organ or tissue in contact with a vascularized site or surface of the host mammal.

Transplantations of organs and tissues or parts thereof, can be performed in an orthotopic, hemi-orthotopic, parallelotopic, or heterotopic manner. An orthotopic transplant, as defined herein, is one in which the donor organ or tissue replaces at least one of the same or homologous structures in the host. A hemi-orthotopic transplant, as defined herein, is one in which the donor organ or tissue replaces one of a pair of the same or homologous structures in the host.

A parallelotopic transplant, as defined herein, is one in which the donor organ or tissue is transplanted so that it receives blood from at least part of the same source of the same or homologous structure in the host. Optionally, the blood drainage of the donor organ or tissue can be to at least one of the same blood vessels as the endogenous host structure or to an at least substantially corresponding vessel. Also optionally, the homologous host organ or tissue that remains in the host can be surgically reduced in size if desired.

A heterotopic transplant, as defined herein, is one in which the donor organ or tissue is transplanted into the host in a location or environment that is not characteristic of the location of the organ or tissue in the donor.

The following examples illustrate surgical techniques for transplanting various organs and tissues to a non-human mammal host, but do not limit the techniques that may be employed for each.

(1) Kidney. The kidney is a paired organ. It is therefore convenient to excise one kidney from the host and transplant the donor kidney, along with a portion of the attached donor ureter, by anastomosing the renal artery and vein of the donor kidney to the abdominal aorta and inferior vena cava, respectively, of the host or to corresponding structures of the host. The donor ureter can be connected to the host bladder, for example, by implanting it in the bladder via a submucosal tunnel. Both kidneys can also be replaced if desired.

(2) Lung. The lung is a paired organ. It is therefore convenient to excise one lung from the host and transplant a donor lung in its place by anastomosis with a pulmonary artery and a pulmonary vein of the host's heart. This step can be performed, for example, by anastomosing a remaining section of pulmonary artery connected to the donor lung to a section of pulmonary artery remaining connected to the host's heart and similarly connecting donor and host pulmonary vein sections. Generally, the transplanted lung should be ventilated in the host to help preserve its structure and function by directly or indirectly connecting it to the trachea (windpipe) or a corresponding structure of the host. However, non-ventilated lung transplants are also within the scope of the invention. If desired or required, a lobe or portion of the remaining host lung can be removed. Both host lungs can also be entirely replaced if desired.

(3) Heart. In a first method, a donor heart is transplanted orthotopically to the host by excising the host heart and anastomosing all of the necessary major arterial and venous blood vessels of the donor heart to at least substantially corresponding vessels of the host. It is well recognized in the art that the functional anatomy of ungulate hearts, and especially that of porcine hearts, is quite similar to that of the human heart. In a second method, a donor heart is transplanted heterotopically to a non-human mammal host, such a sheep or cow, by anastomosing the aorta of the donor heart to the host carotid artery (e.g., end-to-side) and the pulmonary artery of the donor heart the host jugular vein (e.g., end-to-side).

(4) Liver. In a first method, a donor liver is transplanted orthotopically by excising the host liver and anastomosing (i.) a remaining portion of the host hepatic artery to a portion of the donor hepatic artery connected to the donor liver, (ii.) a remaining portion of the host portal vein to a portion of the donor portal vein connected to the donor liver, and (iii.) the donor hepatic veins attached to the donor liver to either the host hepatic veins connected to the inferior vena cava and/or directly to the host inferior vena cava.

In a second method, a donor liver or a part thereof is transplanted parallelotopically with respect to a host liver or a remaining part thereof so that each of the livers receives at least part of the hepatic artery blood and the portal vein blood and each drains directly or indirectly into the inferior vena cava. In a variation, the host portal inflow can be split between the donor and host liver so that the donor liver is provided with intestinal-pancreatic effluent and the host liver with gastric-splenic venous blood. (See, e.g., Lilly et al., Split portal flow in heterotopic hepatic transplantation J Pediatr Surg. 1975 June;10(3):339-48) Those skilled in the art will recognize that a variety of auxiliary liver transplantation techniques are known in the art and can be readily adapted for parallelotopic and heterotopic liver transplantation according to the invention.

(5) Pancreas. In one method, the donor pancreas and a portion of attached duodenum is transplanted to the host while the host pancreas is left in place. The donor pancreatic artery and vein can be joined to the host's iliac artery and vein, respectively. The donor duodenum is joined to the host's small intestine to allow the exocrine enzymes in the main pancreatic duct to enter. In a second related method, the host pancreas is at least partially removed.

(6) Skin. Human skin may be hair-bearing (most of the body, e.g., the scalp) or non-hair-bearing (e.g., palms of hands and soles of feet). Skin consists of three layers (from outside to inside): the epidermis, the dermis (coreum) and a subcutaneous layer comprising areolar and fatty connective tissue. Hair follicles and associated sebaceous (oil) glands are present in hair-bearing skin. In humans, sweat glands are typically present in both hair-bearing and non-hair-bearing skin. A section of human skin including the epidermis and dermis only or the epidermis, dermis and at least part of the subcutaneous layer can be surgically transplanted to a region of a non-human mammal host where the host skin has been at least partly removed. The transplanted skin can be bandaged to ensure good contact with the prepared region of the host to promote the establishment of circulation.

In one embodiment of the invention, growth and expansion of the region of transplanted human skin on the host can be facilitated by selectively injuring or removing the surrounding host skin. For example, this can be performed surgically, physically or chemically using general methods that are restricted to the host skin surrounding the human transplant. An agent that selectively kills host skin cells or retards their growth can also be used, for example, when the non-human mammal host is transgenic for expression of a suicide gene or growth-impairing gene product (see below). Progressive rounds of injury and/or growth retardation of surrounding host skin followed by expansion of the donor skin region may, for example, be employed.

(7) Bone. The blood vessels of bone are numerous. Those of the compact tissue are derived from a close and dense network of vessels ramifying in the periosteum. From this membrane vessels pass into the minute orifices in the compact tissue, and run through the canals which traverse its substance. The cancellous tissue is supplied in a similar way, but by less numerous and larger vessels, which, perforating the outer compact tissue, are distributed to the cavities of the spongy portion of the bone. In the long bones, numerous apertures may be seen at the ends near the articular surfaces; some of these give passage to the arteries of the larger set of vessels referred to; but the most numerous and largest apertures are for some of the veins of the cancellous tissue, which emerge apart from the arteries. The marrow in the body of a long bone is supplied by one large artery (or sometimes more), which enters the bone at the nutrient foramen (situated in most cases near the center of the body), and perforates obliquely the compact structure. The medullary or nutrient artery, usually accompanied by one or two veins, sends branches upward and downward, which ramify in the medullary membrane, and give twigs to the adjoining canals. The ramifications of this vessel anastomose with the arteries of the cancellous and compact tissues. In most of the flat, and in many of the short spongy bones, one or more large apertures are observed, which transmit to the central parts of the bone vessels corresponding to the nutrient arteries and veins. The veins emerge from the long bones in three places: (1) one or two large veins accompany the artery; (2) numerous large and small veins emerge at the articular extremities; (3) many small veins pass out of the compact substance. In the flat cranial bones the veins are large, very numerous, and run in tortuous canals in the diploic tissue, the sides of the canals being formed by thin lamellæ of bone, perforated here and there for the passage of branches from the adjacent cancelli. Gray, Henry. Anatomy of the Human Body. Philadelphia: Lea & Febiger, 1918; Bartleby.com, 2000.

Orthotopic and heterotopic translations of various bones or parts thereof to a host may be made by anastomosing the major arteries and veins of the donor bone to suitable arteries and veins of the host. Depending on the size of the graft, the donor bone may be anastomosed under magnification to the host femoral artery and veins, for example in an end-to-side fashion. See, e.g., Lee et al., Use of swine model in transplantation of vascularized skeletal tissue allografts, Transplantation Proc. (1998) 30, 2743-2745, which is incorporated by reference herein in its entirety.

(8) General sites for heterotopic transplantation include, for example, the kidney capsule, subcutaneous space, and splanchnic vasculature generally. It is well known in the art that the kidney and its capsule provide a highly vascularized environment into which numerous sorts of cells, organs and/or tissues can be heterotopically transplanted and supported in a living state. Subcutaneous transplantation of tissue into a host mammal subcutaneously is also well known in the art. The splanchnic vasculature is recognized as a general site for grafting or otherwise obtaining a circulatory connection between a donor organ or tissue and the host's circulatory system. Accordingly, human cell containing composition, such as a functionally developed human organ or tissue or part thereof can, for example, be heterotopically transplanted under the kidney capsule of a non-human mammal host, transplanted subcutaneously in the host, or grafted or otherwise connected with a host's splanchnic vasculature, in order be supported in a living state by the host

Surgical transplantation techniques for transplanting a human organ or tissue from one human being to another human being are well established. These techniques are readily adaptable for embodiments of the invention in which a human organ or tissue that has been hosted in a non-human mammal is later transplanted back to a human being.

Animal Host Cells Selectively Killable or Separable from Human Cells

Advantageously, the invention also provides embodiments in which at least some of the cells of the host animal are conditionally and selectively “killable,” versus the hosted human cells, in response to a set of one or more conditions. In this manner, the invention provides that host cells that are present in a human organ or tissue or human cell-containing composition that is or was supported by the host can be deleted. For example, the types of host cells that may migrate into and be present in a solid human organ or tissue hosted according to the invention may include, for example, fibroblasts, lymphocytes and/or other immune cells, vascular endothelial cells, and/or host-derived organ or tissue-type specific cells corresponding to the cell or tissue type of the human cells or human cell containing compositions supported by the host. For dispersed tissue types such as bone marrow and blood host cells and hosted human cells may be mixed together, prior to selectively killing the host cells of the mixture and/or separating the human cells from the host cells of the mixture.

Various types of suicide gene strategies can be employed including, but not limited to, the following cases:

Protoxin-to-toxin converting enzyme suicide genes. Examples of suitable converting enzyme type suicide genes include, but are not limited to, thymidine kinase (either wild-type or comprising a mutation), cytosine deaminase, carboxylesterase, carboxypeptidase, deoxycytidine kinase, nitroreductase, guanosine xanthin phosphoribosyltransferase, purine nucleoside phosphorylase, and thymidine phosphorylase. In the absence of the protoxin (prodrug), expression of the suicide gene produces no or little adverse effects on normal cellular metabolism. The product of a converting enzyme type suicide gene acts on a suitable prodrug, converting it into a toxin. In the absence of the suicide gene product, the prodrug is relatively innocuous. Suitable prodrugs for thymidine kinase include ganciclovir, 6-methoxypurine arabinoside, and (E)-5-(2-bromovinyl)-2′ deoxyuridine. A suitable prodrug for cytosine deaminase is 5-fluorocytosine. A suitable prodrug for carboxylesterase is irinotecan. A suitable prodrug for carboxypeptidase is 4-([2-chloroethyl][2-mesyloethyl]amino)benzyol-L-glutamic acid. Suitable prodrugs for deoxycytidine kinase include 4-ipomeanol cytosine arabinoside and fludarabine. Suitable prodrugs for guanosine-xanthin phosphoribosyl transferase include 6-thioxanthine and 6-thioguanine. A suitable prodrug for nitroreductase is 5-aziridin-2,4-dinitrobenzamidine. A suitable prodrug for purine nucleoside phosphorylase is 6-methylpurine deoxyribonucleoside. Suitable prodrugs for thymidine phosphorylase include 5′-deoxy-5-fluorouridine and 1-(tetrahydrofuryl)-5-fluorouracil.

Cell death-inducing suicide genes. Other sorts of suicide genes that can be used according to the invention include that whose gene product, itself, causes or induces cell death. Expression of such a suicide gene and hence cell death can be made conditional by placing expression suicide gene under control of an inducible promoter. One type of cell death inducing suicide gene encodes a protein toxin, e.g., a diphtheria toxin, that kills cells in which it is expressed. Another type of cell death inducing suicide gene encodes an enzyme that acts on cellular substrates to cause or trigger cell death. For example, suitable cell death causing enzyme genes include those encoding cytotoxic proteases such as members of the ICE/CED-3 family of cysteine proteases and caspases, such as Caspase 8h or Caspase 8i (disclosed in U.S. Pat. No. 6,172,190, which is incorporated herein by reference in its entirety).

Signaling-activated suicide gene mechanisms. Transgenic animals engineered so that contacting cells with a dimerizing agent (or clustering agent, generally) activates a signaling pathway causing cell death can also be employed for the present invention. For example, the art provides transgenic animals in which contacting cells with rapamycin or rapalog triggers apoptosis by clustering expressed transgenic fusion proteins that contain intracellular domains of apoptosis mediator molecules, such as the FAS receptor or TNF-R1. Suitable signaling mechanisms are provided, for example, by U.S. Pat. No. 6,649,595, U.S. Pat. No. 6,187,757 and U.S. Pub. No. 20030206891 (application Ser. No. 10/341,967), each of which is incorporated by reference herein in its entirety. In another example, transgenic animals expressing proteins that contain intracellular domains of apoptosis mediator molecules, such as the Fas receptor or TNF-R1 and preselected extracellular epitopes can be used. Divalent or multivalent antibodies recognizing the preselected extracellular epitopes can be contacted with cells expressing these proteins to proximalize (cluster) their intracellular domains and thereby induce apoptosis of the cells.

Negative selection markers generally. In general, any sort of negative selection marker or system that allows or enables the selective killing of non-human mammal host cells of interest can be used according to the invention. For example, where a non-human host mammal either naturally or as a result of genetic modification generally expresses a cell surface epitope that is not expressed by the hosted organ or tissue, cytotoxic agents can be preferentially targeted to the host cells (versus the hosted cells) using antibodies or other binding proteins or molecules that specifically bind the epitope. One or more cytotoxic agents can, for example, be linked directly to such an antibody or binding protein or molecule or an immunoliposome displaying the antibody or binding protein or molecule and containing the cytotoxic agent(s) can be used to shuttle the agent(s) to the target cells.

Promoters. Broad-activity promoters (with respect to cell types) for driving suicide gene expression are those active in many host cell types, at least substantially all host cell types (a universal promoter), or at least active in at least substantially all of the host cell types that are likely to be present in a human organ or tissue that has been supported in the non-human host. Broad activity promoters can be constitutive or inducible.

Suitable broad-activity constitutive promoters include, but are not limited to, the MoMLV LTR, RSV LTR, Friend MuLv LTR, adenovirus promoter, neomycin phosphotransferase promoter/enhancer, late parvovirus promoter, Herpes TK promoter, SV40 promoter, metallothionen IIa gene enhancer/promoter, cytomegalovirus immediate early promoter, and cytomegalovirus immediate late promoter. Suitable broad-activity inducible promoters or inducible expression system can include, but are not limited to, an inducible metallothionein gene promoter, a tetracycline repressor and/or activator based inducible expression system (provided, e.g., by U.S. Pat. Nos. 6,252,136; 6,136,954; 5,912,411; and 5,589,362, each incorporated by reference herein in its entirety); a lac operon based inducible expression system, provided, e.g., by U.S. Pub. No. 20040171824 (application Ser. No. 10/469,881), which is incorporated by reference herein in its entirety; or an ecdysone inducible expression system, provided, e.g., by U.S. Pub. No. No. 20020187972 (application Ser. No. 09/949,278), which is incorporated by reference herein in its entirety. The use of broad-activity regulatory elements, such as a universal promoter, to drive suicide gene expression in the non-human host mammal simplifies deletion of host cells from human cell containing compositions, such as from a human donor organ or tissue, that have been supported in the host. However, the invention also provides that one or a combination of tissue or cell-type specific promoters and regulatory elements generally can also be used. As referred to herein, tissue-specific and cell-type-specific transcriptional regulatory elements, such as promoters and enhancers and combinations thereof, also includes tissue-preferred and cell-type preferred transcriptional regulatory elements.

Numerous suitable tissue-specific and cell-type specific transcriptional regulatory elements are known in the art. The identification and characterization of further tissue-specific and cell-type specific elements, for a given tissue or generally, is a matter of routine research and a common occurrence in the art. Accordingly, the following examples are provided for illustration and in no way limit the invention to only those elements recited herein.

Hepatocyte and/or hepatocytic cell-specific expression can be provided, e.g., by the albumin promoter and, in the fetus, by the alpha-fetoprotein promoter.

Muscle-specific expression can be provided, e.g., by the myosin light chain-2 promoter, alpha actin promoter, troponin 1 promoter, Na.⁺/Ca²⁺ exchanger promoter, dystrophin promoter, creatine kinase promoter, alpha7 integrin promoter, troponin C promoter-enhancer, alpha B-crystallin/small heat shock protein promoter. Cardiac muscle specific expression can be provided, e.g., by the alpha-myosin heavy chain promoter and the atrial natriuretic factor (ANF) promoter.

Endothelial cell-specific expression can be provided, e.g., by gene promoters for the fms-like tyrosine kinase-1 (Flt-1), intercellular adhesion molecule-2 (ICAM-2), von Willebrand factor (vWF), and Vascular Endothelial Growth Factor Receptor-2 (Flk-1). The Fit-1 promoter reportedly directs expression in all vascular beds except those of the liver.

Lung-specific promoters include those for the various lung surfactant proteins, such as the surfactant protein B promoter.

Expression in lymphocytes and/or their progenitors can be provided, e.g., by the jak3 (Janus kinase 3) gene promoter or the LCK gene promoter, which normally drives expression of a lymphocyte specific protein tyrosine kinase.

Kidney-specific expression can be provided, e.g., by the Ksp-cadherin gene promoter or the human PTH/PTHrP receptor gene kidney-specific promoter.

Epidermal cell specific expression can be provided, e.g., by the human epidermal type 1 transglutaminase (TGase I) gene promoter (see U.S. Pat. No. 5,643,746, incorporated by reference herein in its entirety).

Adipose specific expression can be provided, e.g., by the fat-specific promoter/enhancer of the fatty acid-binding protein gene, alpha-P2.

Pancreas-specific expression can be provided, e.g., by the endocrine pancreas-specific insulin promoter (plus or minus the first intron), pancreas alpha-amylase promoters, the pancreas-specific duodenum homeobox 1 (PDX-1) promoter; and the exocrine pancreas-specific promoter of the elastase I gene (Hall et al., J., Biotechnology (1993) 11: 376-379.)

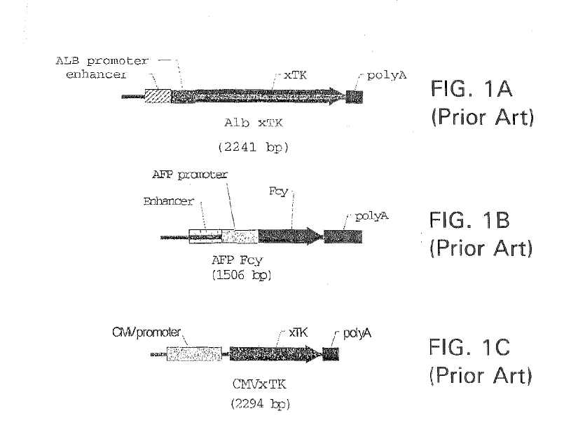

Numerous suicide gene expression constructs and methods including those already described in the art can be employed for providing non-human mammals having conditionally deletable (killable) cells, for use according to the invention. For example, the following sequences and the methods provided by International Publication WO 2004/027029 A2 (PCT/US2003/029251) can be used. SEQ ID NO: 1 provides the sequence of the porcine albumin promoter. SEQ ID NOS: 2-5 provides transgene constructs for the production of transgenic non-human mammals expressing a preselected suicide gene. Specifically, SEQ ID NO: 2 provides a transgene construct including a mutant form of the herpes thymidine kinase suicide gene (xTK) under control of the liver specific porcine Albumin promoter and a poly-A addition signal sequence for the transcript. SEQ ID NO: 3 provides a transgene construct including the suicide gene cytosine deaminase (fCY) under control of the fetal liver-specific alpha-fetoprotein promoter, and a poly-A addition signal sequence for the transcript. SEQ ID NO: 4 provides a transgene construct including a mutant form of the herpes thymidine kinase suicide gene (xTK) under control of a broad-activity, constitutive cytomegalovirus (CMV) promoter, and a poly-A addition signal sequence for the transcript. SEQ ID NO: 5 provides a transgene construct including the suicide gene cytosine deaminase (fCY) under control of a broad-activity, constitutive cytomegalovirus (CMV) promoter, and a poly-A addition signal sequence for the transcript. For illustration, FIG. 1 shows the arrangement of elements of the transgene constructs of SEQ ID NO: 2 (Alb xTK), SEQ ID NO: 3 (AFP Fcy), and SEQ ID NO: 4 (CMV xTK).

xTK is a mutated version of a Herpes simplex virus (HSV) thymidine kinase gene characterized by the substitution of adenosine for cytosine at base positions 130 and 180 The nucleotide substitutions result in a codon changes from leucine to methionine and prevent the phenomenon of male sterility that can occur with the unmodified form. These mutations do not substantially impair enzymatic activity.

The constructs of SEQ ID NOS. 2-5 also include the coding sequence for a form of green fluorescent protein (GFP) under control of a universal promoter. GFP expression allows host cells to be identified visually and easily distinguished from human cells. Such expression of GFP or other markers on host cells may serve as a basis for separating host cells from corresponding human host cells in dispersed tissues such as blood or in mixed suspensions of cells formed from non-dispersed tissues or organs. For example, the expression of fluorescent markers such as GFP permits the host cells and human cells to be separated from each other using a fluorescence-activated cell sorting (FACS) system. Non-human host cells and hosted human cells may also be separated from one another using antibodies that are specific to either the host cells or human cells. For example, human erythrocytes may be separated from mouse host erythrocytes with a human-specific glycophorin-A monoclonal antibody using a magnetic cell separation system such as a CliniMAC^{PLUS} system (Miltenyi Biotec Inc., Auburn, Calif. USA) and/or by FACS.

Example—Production of Transgenic Pigs Expressing a Suicide Gene

This example illustrates the production of transgenic pigs containing a suicide gene expression construct using a somatic cell nuclear transfer technique, as known in the art. Briefly, fibroblasts from 35-day-old fetal pigs are cultured and then transfected with a suicide transgene construct (e.g., either a mutated thymidine kinase or cytosine deaminase construct) using electroporation or any suitable technique. Colchicine is added to arrest the transfected fibroblasts at the G2/M phase. Swine oocytes are isolated and enucleated. For each of several or many enucleated oocytes, a transfected fibroblast is inserted in the perivitelline space using a micromanipulator and electrofusion is then employed to effectively transfer the donor fibroblast nucleus into the enucleated oocyte. Electrofusion and activation can be performed simultaneously or activation can be performed after the electrofusion step. In an alternative method of transfer, the somatic donor nucleus can be microinjected into the enucleated oocyte, followed by activation. In either case, following activation, the reconstructed embryos are implanted into surrogate sows at estrus. The litters can be monitored by ultrasound. At term, the transgenic pigs may be delivered by Caesarean section, if desired.

The presence of the suicide transgene construct(s) in the pigs can be assessed using PCR. Expression of the transgene can be evaluated by Western blotting. The transgenic pigs can be bred once they reach sexual maturity. Pigs homozygous for the suicide gene construct can be obtained by breeding, if desired. Further transgenic pigs can be obtained by breeding and/or cloning.

Suitable dosages for the administration of prodrugs and/or inducers of transcription and/or multimerizing/dimerizing agents can be empirically determined as a matter of routine experimentation. Suitable and optimal dosages may vary with different types of hosts and expression constructs. For example, such agents may be administered to a non-human animal host at a dose of 1-1,000 mg/kg, 1-100 mg/kg, or 5-50 mg/kg. For pigs expressing thymidine kinase, an effective dose of ganciclovir can, e.g., be 1-1,000 mg/kg, 1-100 mg/kg, 5-50 mg/kg, or about 25 mg/kg. For ex vivo killing of non-human host cells present in explanted human donor organs or tissues, a concentration range of 1-1000 mg/l, 1-100 mg/l, 5-50 mg/l or 20-50 mg/l can, for example, be used. For deleting pig host cells expressing thymidine kinase from explanted human donor organs or tissues, ganciclovir concentrations of 2-1000 mg/l, such as about 100 mg/l can, for example, be used. For ex vivo thymidine kinase-based negative selective using the prodrug 5-BrdU (5-Bromo-2′-deoxyuridine), a concentration range of 1-1000 mg/l, 1-100 mg/l, or 25-30 mg/l, can, for example, be used.

In embodiments in which the process of selectively killing host cells is begun or initiated while a human donor organ or tissue still remains in the host, the agent(s) necessary for beginning or initiating such killing can be administered to the host by, for example, intravenous injection. For example, in embodiments where expression of a converting enzyme type of suicide gene is inducible, such induction can be begun before the donor organ(s) or tissue(s) are explanted. The prodrug can then be administered also while the donor organ(s) or tissue(s) remain in the host and/or contacted with the donor organ(s) or tissue(s) after removal from the host.

For ex vivo killing of host cells, the agents necessary for negative selection can be prepared in liquid media that is contacted with the explanted organ or tissue, for example, by immersion in such media and/or by perfusion with such media.

Reducing Transfer of Xenoantigens from a Host

Certain embodiments of the invention are based on the recognition that cell surface antigens of a non-human mammal host can be transferred, by natural mechanisms, to the cell surface of foreign donor cells (e.g. human) and/or donor extracellular matrix (e.g. human) that are resident within the host mammal. One embodiment of the invention provides for reducing the expression of at least one xenoantigen in a non-human mammal host in order to reduce or eliminate its transfer to foreign donor cells, organs and/or tissues that are resident in and supported by the non-human mammal host. For example, the transfer of major xenoantigens for humans, such as alpha-galactosyl epitopes, and/or minor xenoantigens for humans can be reduced.

As referred to herein, glycosylphosphatidylinositol-anchored. proteins are proteins bound to the lipid bilayer of a membrane through either a glycosylphosphatidylinositol anchor (GPI-anchor), which is a complex oligoglycan linked to a phosphatidylinositol group, or a GPI-like-anchor, i.e., a similar complex oligoglycan linked to a sphingolipidinositol group, resulting in the attachment of the C-terminus of the protein to the membrane. Certain extracellular carbohydrate epitopes are also directly linked to cell membrane lipids.

Glycosylphosphatidylinositol (GPI) anchored proteins are known to be exchanged between the membranes of living cells in vivo, for example, from erythrocytes to endothelium and vice versa. Medof et al., Cell-surface engineering with GPI-anchored proteins, Cell Surface Eng'g (1996) Vol. 10, pp. 574-586; Kooyman et al. (1995) In vivo-transfer of GPI-linked complement restriction factors from erythrocytes to endothelium Science, Vol. 269, pp. 89-92. GPI-anchored proteins are also known to be exchanged between the membranes of erythrocytes. Sloand et al. (2004) Transfer of glycosylphosphatidylinositol-anchored proteins to deficient cells after erythrocyte transfusion in paroxysmal nocturnal hemoglobinuria Blood (12):3782-3788. See also: Babiker et al. (2005) Transfer of functional prostasomal CD59 of metastatic prostatic cancer cell origin protects cells against complement attack Prostate. 62(2):105-114; Dunn et al. (1996) A knock-out model of paroxysmal nocturnal hemoglobinuria: Pig-a(−) hematopoiesis is reconstituted following intercellular transfer of GPI-anchored proteins Proc Natl Acad Sci USA. 93(15):7938-7943; and Anderson et al. (1996) Intercellular transfer of a glycosylphosphatidylinositol (GPI)-linked protein: release and uptake of CD4-GPI from recombinant adeno-associated virus-transduced HeLa cells Proc Natl Acad Sci USA. 93(12):5894-5898. Proteins that are loosely embedded in the cell membrane, such as those with short tails embedded in, but not traversing the cell membrane, may also be subject to intercellular transfer by natural mechanisms. The present invention is not limited by the mechanism of intercellular transfer.

In embodiments of the invention in which donor cells, e.g., in the form of organs or tissues, are to be supported in a living state by a host mammal and later transplanted or transferred to a recipient mammal, such as a human patient, antigens transferred from the host mammal to the donor material can contribute to immunological rejection of the donor material by the recipient. Such xenoantigens can, for example, include peptide epitopes of transferred proteins and/or carbohydrate epitopes present on the transferred proteins, such as alpha-galactosyl epitopes and N-glycolyineuraminic acid (NeuGc) epitopes, as well as carbohydrate epitopes directly linked to cell membrane lipids or otherwise linked to the cell membrane.

Alpha-Galactosyl Epitopes

In the case where the host mammal is of the type that produces alpha-galactosyl (Gal.alpha.(1,3)Gal) epitope modified proteins, such as an ungulate or rodent, and the hosted cells comprise alpha-galactosyl epitope negative cells (i.e., cells not producing alpha-galactosyl epitopes), such as human cells, the invention provides for reducing or eliminating completely the amount of alpha-galactosyl epitopes transferred to the epitope-negative cells by employing a non-human mammal host modified to reduce or completely eliminate the expression of alpha-galactosyl epitopes.

Numerous methods for producing genetically modified animals having reduced expression of alpha-galactosyl epitopes are known in the art including: (1) genetic knock-out of the Gal.alpha.(1,3) galactosyl transferase gene (“alpha-galactosyl transferase gene;” GGTA1) by homologous recombination; (2) expression of transgenes encoding other transferases, such as alpha-fucosyltransferase (e.g., human FUT1 and/or FUT2), that compete with alpha-galactosyltranferase for substrate; and (3) expression of transgenes encoding human N-acetylglucosaminyltransferase III which reduces formation of alpha-galactosyl epitopes by inhibiting N-linked sugar branching. Genetically-modified non-human mammals with reduced alpha-galactosyl epitope expression and methods for producing them are provided, for example, by the following patents or applications, each of which is incorporated by reference herein in its entirety: U.S. Pat. No. 6,413,769; U.S. Pat. No. 6,331,658; U.S. Pat. No. 6,166,288; U.S. Pat. No. 5,821,117; U.S. Pat. No. 5,849,991; U.S. Pub. No. 20040268424 (application Ser. No. 10/646,970); U.S. Pub. No. 20030203427 (application Ser. No. 10/125,994); U.S. Pub. No. 20030068818 (application Ser. No. 10/105,963); U.S. Pub. No. 20020031494 (application Ser. No. 10/254,077); U.S. Pub. No. 20030014770 (application Ser. No. 10/098,276) U.S. Pub. No. 20040073963 (application Ser. No. 10/362,429); U.S. Pub. No. 20040171155 (application Ser. No. 10/762,888); and U.S. Pub. No. 20030131365 (application Ser. No. 10/172,459). Mice homozygously deficient for the GGTA1 gene and method for making the same are, for example, provided by U.S. Pat. No. 5,849,991. Swine homozygously deficient for the GGTA1 gene and methods for making the same are, for example, provided by U.S. Pub. No. 20040268424. SEQ ID NO: 6 provides the mRNA sequence of a porcine alpha(1,3)galactosyl tranferase gene (GGTA1). Sheep and cow mRNA sequences for the GGTA1 gene are provided in GenBank accession nos. NM_{—}001009764 [SEQ ID NO: 7] and NM_{—}177511 [SEQ ID NO: 8], respectively. The mouse mRNA sequence for the GGTA1 gene is provided, for example, in Genbank accession no. NM_{—}010283 [SEQ ID NO: 9].

Isogloboside 3 (iGb3) synthase is another enzyme that, in addition to alpha(1,3)-galactosyltransferase, synthesizes Galα(1,3)Gal motifs. In contrast to alpha(1,3)-galactosyltransferase, iGb3 synthase preferentially modifies glycolipids over glycoprotein substrates. (Keusch et al. (2000) Cloning of Gb3 synthase, the key enzyme in globo-series glycosphingolipid synthesis, predicts a family of alpha 1,4-glycosyltransferases conserved in plants, insects, and mammals J. Bio. Chem. 275:25308-25314.) iGb3 synthase acts on lactosylceramide (LacCer (Gal.beta.1,4Glc.beta.1 Cer)) to form the glycolipid isogloboid structure iGb3 (Gal.alpha.1,3Gal.beta.1,4Glc.beta.1-Cer), initiating the synthesis of the isoglobo-series of glycoshingolipids. Genetically-modified swine having reduced or eliminated expression of iGb3 synthase and methods and sequences for producing the same are provided, e.g., by U.S. Pub. No. 20050155095 (application Ser. 10/981,935), which is incorporated by reference herein in its entirety. The mRNA sequence of the rat iGb3 synthase gene has been reported in GenBank accession no. NM_{—}138524 [SEQ ID NO: 10] and that of the mouse gene in GenBank accession no. NM_{—}001009819 [SEQ ID NO: 11].

According to the invention, the expression of a selected enzyme such as alpha-galactosyl transferase (GGTA1), iGB3 synthase or CMP-NeuAc hydroxylase or a protein xenoantigen may be reduced or completely eliminated in a non-human mammal host (or non-human mammal or organ or tissue thereof generally) by post-transcriptional silencing employing dsRNA (RNA interference, RNAi) and/or transcriptional gene silencing employing dsRNA and/or by antisense methods. Double-stranded RNA molecules used for such silencing may be produced within cells of the host from the host genome or from a vector introduced into the cells and/or may be exogenously provided to the cells. Any of the forms of dsRNA that induce post-transcriptional silencing and/or transcriptional gene silencing can be used including, but not limited to, siRNA (e.g., digestion products of an RNAse III such as Dicer or similarly sized and configured short dsRNA molecules), short hairpin RNA, and designed microRNA (mRNA). The design and selection of effective molecules and strategies for RNA silencing and antisense regulation of preselected targets is well established in the art. Nucleotide sequences for porcine alpha-galactosyl transferase are provided, for example, by U.S. Pat. No. 5,849,991, U.S. Pat. No. 5,821,117 and U.S. Pub. No. 20030203427, each of which is incorporated by reference herein in its entirety.

It should further be understood that the reduction and/or elimination of xenoantigens and/or xenoantigen-producing enzymes can be, but is not necessarily, performed prior to the introduction of the human cells into the non-human mammal host. For example, human cells may be introduced into a fetal non-human mammal host to integrate into one or more organs and tissues of the host and following birth of the host, the reduction or elimination of a xenoantigens and/or xenoantigen-producing enzyme may be induced by any method, such as treatment with RNA silencing molecules that silence expression of a xenoantigen or a xenoantigen-producing enzyme. A transgenic host may also be provided in which RNA silencing of a xenonatigen or xenoantigen-producing enzyme can be induced and/or in which the expression of an enzyme that cleaves or interferes with production of a xenoantigen can be induced.

Alpha-galactosyl epitopes expressed on host cells that could be transferred to cells that do not express such epitopes, as well as alpha-galactosyl epitopes already transferred to cells that do not express alpha-galactosyl epitopes, can also be removed enzymatically, for example, by alpha-galatosidase or endo-beta-galactosidase C. Such enzymes can, for example, be infused intravenously into a host supporting a human donor organ or tissue and/or expressed constitutively or inducibly in a suitable host. Enzymatic removal of alpha-galactosyl epitopes is taught, for example, by U.S. Pat. No. 6,758,865; U.S. Pat. No. 6,491,912; U.S. Pat. No. 6,331,319; U.S. Pat. No. 6,046,379, and Maruyama et al., Xenotransplantation 11 (5), pp. 444-51 (2004), each of which is incorporated by reference herein in its entirety.

N-Glycolyineuraminic Acid (NeuGc) Epitopes

N-acetyineuraminic acid (NeuAc) and N-glycolyineuraminic acid (NeuGc) are abundant forms of sialic acid that are found as cell surface carbohydrate modifications to proteins and lipids. NeuGc is present in most animals with the notable exception of humans and chickens. Thus, NeuGc is a xenoantigen with respect to the human immune system. NeuGc is synthesized in vivo from N-acetyineuraminic acid (NeuAc) by the addition of a single hydroxyl group by cytidine monophospho-N-acetyineuraminic acid hydroxylase (CMP-NeuAc hydroxylase). According to the present invention, non-human mammals, or organs or tissues thereof, with reduced or completely eliminated expression of NeuGC epitopes can be produced by any suitable method such as (i.) genetic knockout of the CMP-NeuAc hydroxylase gene by homologous recombination, (ii.) post-transcriptional RNA silencing, dsRNA-mediated gene silencing of transcription, and/or antisense techniques against the CMP-NeuAc hydroxylase gene, and/or (iii.) enzymatic removal of NeuGc epitopes using a suitable enzyme such as neuramimidase. Neuramimidase removes both NeuGc and NeuAc cell surface epitopes. If desired, the NeuAc epitope can be regenerated by further treatment with sialyltransferase, using cytidine monophospho-N-acetyineuraminic acid (CMP-NeuAc) as a substrate. The production of non-human mammals genetically modified to eliminate CMP-NeuAc hydroxylase gene expression and nucleotide sequences required therefor, as well as methods for enzymatic removal of NeuGC epitopes are provided by U.S. Pub. No. 20030165480 (application Ser. No. 10/135,919), which is incorporated by reference herein in its entirety. See also International Pub. No. WO 2004/108904. SEQ ID. NO. 12 is a partial mRNA coding sequence of the porcine CMP-NeuAc hydroxylase gene, derived from U.S. Pub. No. 20030165480. The mRNA sequence of the major and minor alternatively spliced forms of the mouse CMP-NeuAc hydroxylase gene are provided by Genbank accession nos. ABO61276 [SEQ ID NO: 13] and ABO61277 [SEQ ID NO: 14], respectively.

Animal hosts characterized by reductions or complete elimination in both alpha-galactosyl epitopes and NeuGc epitopes can also be used according to the invention. In one embodiment of the invention, a double gene knock-out, non-human mammal, for example an ungulate or rodent that is homozygously negative for both alpha-galactosyltransferase and CMP-NeuAc hydroxylase is used as a non-human mammal host. Heterozygous knockouts are also within the scope of the invention. In another embodiment, the non-human mammal host has either or both of the alpha-galactosyltransferase gene and CMP-NeuAc hydroxylase gene knocked-out (homozygously), and includes a transgene directing the expression of at least one tolerance-promoting biomolecule. As an alternative to genetic deletions or disruption by homologous recombination, genetic knock-outs used according to the invention may also be conditionally obtained, optionally in a tissue-specific manner, using, for example, inducible recombinase expression methods and systems, such as the CRE-LOX system, for gene deletion, as known in the art.

In addition to the intercellular transfer of xenoantigens from host cells to hosted human cells, host xenoantigens can, at least in some instances, also be present in a hosted human cell containing composition, such as an organ or tissue, in the form of living or dead host cells and/or fragments, such as cell membrane fragments, thereof. Accordingly, one embodiment of the invention provides a method for causing hosted human cell containing compositions such as organs or tissues to be better tolerated upon retransplantation to a human recipient by using a non-human mammal host that is genetically modified to decrease or completely eliminate the expression of at least one xenoantigen that is not intercellularly transferable from host to donor cells, for example a xenoantigenic host transmembrane protein. Examples of xenoantigenic transmembrane proteins, with respect to a human recipient immune system, include at least some non-human, transmembrane MHC class I and MHC class II molecules.

Transfer of Tolerance Promoting Biomolecules from Host to Donor Organ or Tissue

Another aspect of the invention provides a non-human host mammal modified to express or increase its expression of at least one transferable “tolerance promoting” biomolecule that, when transferred to foreign donor cells (human and/or non-human) resident in the host mammal, improves the tolerability of the donor cells to the immune system of a preselected type of intended recipient, such as a human.

Methods for expressing selected GPI-anchored proteins are well established. For example, several complement inhibiting factors such as human or non-human forms of DAF (decay accelerating factor; CD55), CD59 (membrane inhibitor of reactive lysis, MIRL) and MCP (membrane cofactor protein, CD46) occur in GPI-anchored forms. These complement inhibitors are found, for example, on red blood cells and the endothelium, which is a critical site for immunological tolerance or rejection. In one embodiment of the invention, transgenic non-human host mammals expressing or having increased expression of (versus normal endogenous expression) a human or non-human form of at least one of these GPI-anchored complement inhibitors is employed as an animal host for human organs or tissues. A related embodiment provides that expression of at least one tolerance-promoting biomolecule, such as a protein, that is not naturally present, or increased expression of a tolerance-promoting biomolecule that is naturally present, by a non-human mammal host, such as but not limited to at least one of the listed complement inhibitors, results in transfer or increased transfer of the tolerance promoting biomolecule(s) to at least some of the foreign donor cells resident in host mammal.

Further, methods for expressing the extracellular domain, or one or more selected portions thereof, of a selected protein that is not regularly expressed in a GPI-anchored form, as a GPI-anchored protein or a GPI-anchored fusion protein are well established in the art and can be used according to the invention to create transgenic non-human mammal hosts in which selected tolerance-promoting transgene products are transferable to foreign donor cells resident in the host.

GPI-anchored proteins, like other membrane-associated proteins, are modified by the addition of carbohydrate moieties. For example, human CD59 has a single N-glycosylation site and a number of potential O-glycosylation sites. Rudd et al. The glycosylation of the complement regulatory protein, human erythrocytes CD59. (1997) J. Biol. Chem., 272, 7229-7244. Accordingly, one embodiment of the invention provides a non-human host mammal that is genetically modified to express a transferable tolerance promoting biomolecule, such as hCD59, and which also has reduced expression of at least one carbohydrate xenoantigen, such as alpha-galactosyl or NeuGc epitopes, e.g., as the result of genetic modification. Such a host can be used, in any manner described, to support human cells, such as organs and tissues, in a living state. Advantageously, the use of such a host prevents the modification of tolerance-promoting biomolecules, such as tolerance-promoting proteins, with undesirable carbohydrate xenoantigens and thus, prevents their transfer to the hosted human organs or tissues while improving the tolerance promoting effect of the transferred tolerance-promoting biomolecule(s). Another embodiment provides a method including the steps of hosting human cells, for example, at least part of a human organ or tissue, in a living state in a non-human mammal host that is genetically modified to express at least one transferable tolerance-promoting biomolecule that is subject to in vivo glycosylation and thereafter enzymatically treating the human organ or tissue, for example, after explanation, to remove carbohydrate xenoantigens, such as those transferred from the host to the human organs or tissues, for example, those attached to the tolerance-promoting biomolecule(s).

The following examples illustrate various tolerance-promoting biomolecules suitable for expression in transgenic mammal hosts according to this aspect of the invention and/or provide such hosts.

(i.) U.S. Pat. No. 6,825,395 and U.S. Pub. No. 20030165480, each incorporated by reference herein in its entirety, provide transgenic non-human mammals expressing hDAF.

(ii.) U.S. Pat. No. 6,639,122, incorporated by reference herein in its entirety, provides transgenic swine expressing HLA-D.

(iii.) Transgenic mammals expressing membrane-tethered fusion protein forms of one or both of the anticoagulants human tissue factor pathway inhibitor and hirudin can be used. See Chen et al., Complete inhibition of acute humoral rejection using regulated expression of membrane-tethered anticoagulants on xenograft endothelium. Am J Transplant. 2004 December; 4(12):1958-63 and U.S. Pat. No. 6,423,316, each of which is incorporated by reference herein in its entirety.

(iv.) Transgenic mammals expressing human HLA-G to protect from lysis by human NK cells can be used. Human natural killer (NK) cells, which can directly lyse porcine endothelial cells, play an important role in xenotransplantation. HLA-G is a nonclassical major histocompatibility complex (MHC) class I molecule that has been implicated in protecting susceptible target cells from lysis by NK cells. Wang et al., A study of HLA-G1 protection of porcine endothelial cells against human NK cell cytotoxicity. Transplant Proc. 2004 October; 36(8): 2473-4.

(v.) Transgenic mammals expressing cell surface human Fas ligand, which induces apoptosis of Fas Receptor bearing cells, can be used. Rodriguez-Gago et al., Human anti-porcine gammadelta T-cell xenoreactivity is inhibited by human FasL (Fas ligand) expression on porcine endothelial cells, Transplantation. 2001 Aug. 15; 72(3):503-9. Since human cells of a human donor organ or tissue that has been supported in a non-human mammal host can appear non-human to the immune system of a human recipient due to transferred host antigens, the invention also provides that the host can express human FasL that can be transferred to the human donor organ or tissues in order to limit immune rejection against the human cells upon further transplantation to a human being. FasL naturally occurs as a transmembrane protein. According to the invention, the extracellular domain of human FasL, such as amino acids Leu 107 to Leu 281, can also be expressed as a GPI-anchored protein or fusion protein, in a monomeric or multimeric form, either constitutively or inducibly, in a transgenic non-human host mammal.

As described above, in addition to the intercellular transfer of xenoantigens from host cells to foreign donor cells, host xenoantigens can, at least in some instances, also be present in a hosted human cell containing composition, such as a human organ or tissue, in the form of living or dead host cells and/or fragments, such as cell membrane fragments, thereof. Accordingly, one embodiment of the invention provides a method for causing hosted human cell containing compositions, such as human organs or tissues, to be better tolerated upon retransplantation to a human recipient by using a non-human mammal host that is genetically modified to express or increase the expression of at least one tolerance-promoting biomolecule, which is or is not intercellularly transferable from host to donor cells. In this manner, the tolerance-promoting biomolecule(s) at least partially ameliorates recipient immune reactions to xenoantigens present on the host cells or fragments and thereby reduces the general recruitment of a negative immune response toward the human organ or tissue in a human recipient. Host cell fragments may be present in a human cell containing composition, such as a donor human organ or tissue, even after host cells therein have been selectively killed if they have not had time to clear and/or have not been actively cleared. Methods for clearing hosted human organs and tissues of host antigens and cellular material are provided by further embodiments described below.

Post-Conditioning Embodiments

A further aspect of the invention provides methods for conditioning human cell containing compositions, such as human organs and tissues, which have been supported in a living state in a non-human mammal host to be better tolerated by the immune system of a preselected type of recipient, such as a human being. In one embodiment, a hosted human organ or tissue is isolated from the mammal host's circulation, for example, by explantation from the host, and is at least partially cleared of xenogeneic (with respect to the preselected type of recipient, such as a human) cells, xenogeneic cellular material, xenogeneic extracellular material and/or xenogeneic antigens that may be present in the organ or tissue. In a related embodiment, the treated organ or tissue is then transplanted to the preselected type of recipient, such as a human.

In one embodiment, major and/or minor xenoantigens from the non-human mammal host that are present within the hosted human cell containing composition, for example membrane-linked proteins and/or carbohydrate epitopes that were transferred from the host to the human cell containing composition or cellular debris of host cells are, at least in part, passively cleared from the organ or tissue after isolation from the mammal host's circulation as a result of their natural turnover and degradation.

In another embodiment, the removal of xenogeneic material from the hosted human cell containing composition is actively facilitated after isolation from the mammal host. In one case according to the invention, the cells of the non-human mammal host are selectively killable over the cells of the host mammal and the hosted human cell containing composition, such as a human organ or tissue, is subjected to the conditions required to selectively kill unwanted mammal host cells that were resident in the product organ or tissue, e.g., by contacting the human cell containing composition with the necessary agent(s). The cellular debris that result from this killing process may, for example, be at least partially cleared from the human cell containing composition by perfusion of the composition, for example where the composition is an organ or tissue, after isolation from the mammal host. They may also be optionally filtered out of the perfusate, for example, in the case where the perfusate recirculates through the composition.

In another embodiment, xenogeneic cell surface antigens that may be present within a human cell containing composition, such as a human organ or tissue, are actively removed or modified enzymatically after isolation from the mammal host by contacting the composition with a medium containing enzymes, for example, by immersion in or perfusion with the medium. For example, the invention provides that carbohydrate xenoantigens that may be present in a human organ or tissue can be removed by perfusing the organ or tissue with a medium containing an appropriate glycosidase, such as an alpha-galactosidase or endo-beta-galactosidase C (EndoGalC) for removing alpha-galactosyl epitopes and/or neuramimidase for removing NeuGc epitopes. (Alpha-gal: U.S. Pat. No. 6,758,865; U.S. Pat. No. 6,491,912; U.S. Pat. No. 6,331,319; U.S. Pat. No. 6,046,379 and Maruyama et al. Xenotransplantation. 2004 September; 11 (5): 444-51; NeuGc: U.S. Pub. No. 2003/0165480 (application Ser. No. 10/135,919), each of which is incorporated by reference herein in its entirety.) These particular epitopes may be present when the non-human mammal used as a host has not been genetically modified to eliminate their expression.

Similarly, one aspect of the invention provides that GPI-anchored major or minor xenoantigens (the proteins or xenoantigenic moieties linked to the GPI-anchored proteins) can be at least partly removed by generally removing GPI-linked proteins by contacting (e.g., by immersion or perfusion) the composition (e.g., organ or tissue) with a suitable enzyme such as a phosphatidylinositol-specific phospholipase C(PI-PLC) or phosphatidylinositol-specific phospholipase D (PI-PLD). Suitable phospholipases are provided, for example, by U.S. Pat. No. 6,689,598; U.S. Pat. No. 6,638,747; and U.S. Pat. No. 5,418,147, each of which is incorporated by reference herein in its entirety. Advantageously, GPI-anchored xenoantigens arising from the mammal host are thus cleared, while removed GPI-anchored biomolecules specific to the human cells will be naturally regenerated.

Another embodiment includes initiating or at least partly performing the cell-death inducing treatment or enzymatic treatments described above while the human cells, such as a human organ or tissue, are not yet isolated from the mammal host's circulation. The human cells can then be isolated from the mammal host before the effects of the treatment are substantially undone by further contact with the host circulatory system.

Methods and media for perfusing organs and tissue are well developed in the art. Suitable methods and media are provided, for example, by U.S. Pat. No. 6,699,231; U.S. Pat. No. 6,677,150; U.S. Pat. No. 6,680,305; U.S. Pat. No. 6,627,393; U.S. Pat. No. 6,589,223; U.S. Pat. No. 6,506,549; U.S. Pat. No. 6,589,223; U.S. Pat. No. 6,677,150; U.S. Pat. No. 6,589,223; U.S. Pat. No. 6,524,785; U.S. Pat. No. 6,100,082; U.S. Pat. No. 5,965,433; U.S. Pat. No. 5,586,438; U.S. Pat. No. 5,498,427 U.S. Pat. No. 5,599,659; U.S. Pat. No. 6,492,103 and U.S. Pat. No. 5,362,622, each of which is incorporated by reference herein in its entirety.

Extra-Corporeal Support

A related embodiment of the invention includes the steps of explanting a hosted human solid organ or tissue or part thereof from the non-human mammal host in which it was supported and thereafter supporting the organ or tissue in a living state in isolation from the non-human mammal host using an extracorporeal support device and/or method, for a period of time. During the period of extracorporeal support, at least some xenogeneic (with respect to the preselected type of recipient, such as a human) cells, xenogeneic cellular material, xenogeneic extracellular material and/or xenogeneic antigens, from the non-human host mammal are actively and/or passively removed (cleared) from the organ or tissue in, for example, the same manners described above. In a related embodiment, the treated organ or tissue is transplanted to the preselected type of recipient after the period of extracorporeal support. In one embodiment, the period of extracorporeal support is approximately 1, 2, 3, 4, 5, 6, 7 or 14 days. In another embodiment, the period of extracorporeal support is at least 1, 2, 3, 4, 5, 6, 7 or 14 days.

Any type of extracorporeal device and/or method for the support of living donor organs or tissues can be used. Some of these devices are similar to heart-lung machines in that they perfuse the subject organ or tissue with a medium providing oxygen and nutrients. This medium may, for example, be based at least in part on blood and/or artificial blood, such as a hemoglobin-based blood substitute or a fluorocarbon based blood substitute. One such device is the Transmedics Portable Organ Preservation System (POPS). Suitable extracorporeal support devices and/or methods include, but are not limited to those described in, U.S. Pub. No. 20040171138; U.S. Pat. No. 6,100,082; U.S. Pat. No. 6,046,046; U.S. Pat. No. 6,677,150; U.S. Pat. Nos. 6,673,594; 6,642,045; U.S. Pat. No. 6,582,953; U.S. Pat. Nos. 6,794,182; 5,326,706; U.S. Pat. No. 5,494,822; U.S. Pat. No. 4,837,390; U.S. Pat. No. 4,186,565; U.S. Pat. No. 4,745,759; and U.S. Pat. No. 5,807,737 each of which is incorporated by reference herein in its entirety.

One extracorporeal support embodiment provides a method that includes the steps of: explanting an human organ or tissue or part thereof from a non-human mammal host in which the organ or tissue or part thereof was maintained in a living state, thereafter maintaining the organ or tissue in a living state on extracorporeal support for a period of time, and during at least part of the period of extracorporeal support, selectively killing non-human host cells and/or enzymatically treating the organ or tissue to remove xenoantigens, as described above. A related method further includes the step of: after the period of extracorporeal support, transplanting the human organ or tissue or part thereof to a human recipient.

Another extracorporeal support embodiment provides a method that includes the steps of: initiating or at least partly performing the selective deletion of host cells from an human solid organ or solid tissue or part thereof and/or enzymatically treating the organ or tissue or part thereof to remove xenoantigens, as described above, while the organ or tissue or tissue is not yet isolated from the mammal host's circulation; explanting the organ or tissue before the effects of the treatment(s) are substantially undone by further contact with the host circulatory system; and thereafter maintaining the organ in a living state on extracorporeal support. One or more of the treatments described can also be performed or continued during support of the organ or tissue or part thereof by the extracorporeal support device. A related method embodiment further includes the step of: after the period of extracorporeal support, transplanting the human organ or tissue or part thereof to a recipient, such as a human recipient.

Further examples of the invention are provided as follows:

Example—Selectively Advantaged Growth of Human Liver Cells in the Liver of a Transgenic, Xenoantigen-Reduced Non-Human Mammal Host

A genetically immunocompromised, GGTA1^{-/-} (alpha(1,3)galactosyltransferase null) mouse comprising a transgene that provides for liver-specific production of urokinase-type plasminogen activator (uPA), for example driven by the Albumin promoter (an Alb-uPA transgene) is provided. The production and use of SCID mice hemizygous and homozygous for Alb-uPA for the selective repopulation of mouse livers with human hepatocytes has been described in U.S. Pub. No. 20030115616, which is incorporated by reference herein in its entirety. The GGTA1−/− modification may be introduced by any method, for example, by knocking out one copy of the GGTA1 gene in SCID/Alb-uPA mice using homologous recombination and selectively breeding progeny to produce homozygous GGTA1−/− SCID Alb-uPA mice. 4-12 day-old, such as 7 day-old, GGTA1−/− SCID Alb-uPA progeny are transplanted intrasplenically with 0.5-1×0.10⁶ freshly isolated viable human hepatocytes. Intrasplenically injected hepatocytes rapidly translocate to the liver via the portal venous system and engraft into the parenchyma surrounding terminal portal venules. Since the uPA transgene has a growth-retarding and viability-impairing effect on the native mouse hepatocytes, population and expansion of the mouse liver with the human hepatocytes is selectively advantaged.

Example—Establishment of Human Erythropoiesis in a Xenoantigen-Reduced Non-Human Mammal Host by Transplantation of Human Bone

An at least substantially immunotolerant non-human mammalian host modified or treated to reduce the expression of at least one xenoantigen (with respect to human tolerance of xenogeneic tissue) such as alpha-galactosyl epitopes, such as alpha(1,3)galactosyl-transferase (GGTA1)-synthesized alpha-galactosyl epitopes, is provided. The host may, for example, be a Rag1^{null} pig that is also GGTA1^{null}. Human hematopoiesis, such as erythropoiesis, is established in the host by transplanting at least part of a red-marrow-containing human bone, such as a vertebrae, rib, sternum, pelvis, femur (containing red marrow), humerus and skull, to the host to be supported in a living state therein. Erythropoiesis may be stimulated by administering erythropoietin to the host. Transplantation may be effectuated by anastomosing the major arteries entering and veins leaving the bone with suitable host arteries and veins. For example, where the donor bone is a human rib, a host rib may be removed so that anastomoses with the arteries and veins supplying and draining the explanted host rib can be made. Alternatively, and depending on the size of the graft, the donor bone may be anastomosed under magnification to the host femoral artery and veins, for example in an end-to-side fashion, as in Lee et al., Transplantation Proc. (1998) 30, 2743-2745.

Example—Development of Functional Human Blood, Including Erythrocytes and Platelets, and Human Immune Systems in a Xenoantigen-Reduced Non-Human Mammal Host

The development of functional human blood, including erythrocytes and platelets, and human immune systems in NOD/SCID/IL2 Receptor y Chainnull mice has been previously described in Ishikawa et al., Development of functional human blood and immune systems in NOD/SCID/IL2 receptor {gamma} chain(null) mice Blood (2005) 106(5), 1565-1573, which is incorporated by reference herein in its entirety. In addition to the SCID phenotype, these mice have deficiencies in NK cell activity and innate immunity resulting from the IL2R γ^{null} defect. The NOD/SCID/IL2 Receptor γ Chain^{null} mouse line was developed at the Jackson Laboratory (Bar Harbor, Me.) and is described in Shultz et al., Human lymphoid and myeloid cell development in NOD/LtSz-scid IL2R gamma null mice engrafted with mobilized human hemopoietic stem cells J. Immunology (2005) 174(10), 6477-6489, which is incorporated by reference herein in its entirety. Briefly, the double mutant mice were produced by the developer by breeding female NOD.CB17-Prkdc^{scid}/J (Jackson Laboratory Stock #1303) mice with male mice bearing the X-linked B6.129S4 //2rg^{tm1Wjl}/J allele (Jackson Laboratory Stock #3174). The resulting male mice heterozygous for the Prkdc^{scid} allele and hemizygous for the II2 rg^{tm1Wjl} allele were crossed to female NOD.CB17-Prkdc^{scid}/J (Jackson Laboratory Stock #1303) mice for 8 generations. Heterozygotes were interbred to produce mice homozygous for the Prkdc^{scid} allele and homozygous (females) or hemizygous (males) for the //2rg^{tm1Wjl} allele. The mice can be reproduced from the available stock lines according to this method or can be directly purchased from The Jackson Laboratory (as Stock #4048) subject to the consent of the developer.

According to this example of the present invention, the NOD/SCID/IL2 Receptor γ Chain^{null} mouse line is further modified to eliminate expression of the GGTA1 gene so that alpha(1,3)galactosyltransferase (GGTA1)-mediated synthesis of alphagalactosyl epitopes no longer occurs. For example, a GGTA1^{-/-} (null) modification may be introduced by any method, such as by knocking out one copy of the GGTA1 gene in NOD/SCID/IL2 Receptor γ Chain^{null} mouse line by homologous recombination and selectively breeding progeny to produce GGTA1 ^{null}/NOD/SCID/IL2 Receptor γ Chain^{null} mice.

Human cord blood (CB) cells are obtained, for example, according to standard procedures upon obtaining consent. Mononuclear cells are depleted of Lin⁺ cells using mouse anti-hCD3, hCD4, hCD8, hCD11 b, hCD19, hCD20, hCD56, and human glycophorin A (hGPA) monoclonal antibodies (BD Immunocytometry, San Jose, Calif.). Samples are also enriched for hCD34+ cells by using anti-hCD34 microbeads (Miltenyi Biotec Inc., Auburn, CA). These cells are further stained with anti-hCD34 and hCD38 antibodies (BD immunocytometry), and purified for Lin−CD34+CD38− HSCs using a FACSVantage (Becton Dickinson, San Jose, Calif.). The Lin⁻hCD34⁺ CB fraction contains early myeloid and lymphoid progenitors as well as HSCs. 1×10⁵ Lin⁻ hCD34⁺ cells or 2×10⁴ Lin⁻hCD34⁺hCD38⁻ cells are transplanted into irradiated (100 cGy) GGTA1^{null}/NOD/SCID/IL2rγ^{null} via a facial vein within 48 hrs of birth. The human cells are then allowed to develop within the mouse host.

As reported in Ishikawa et al., Blood (2005) 106(5), 1565-1573, at three months following transplantation of the human cells into the mouse host, human Glycophorin A⁺ erythrocytes and human CD41a⁺ platelets are circulating and being produced in the bone marrow. Moreover, various other human cell types are produced by following the method. In the bone marrow and the spleen, CD11c⁺ dendritic cells as well as hCD33⁺ myeloid cells, hCD19⁺ B cells, and hCD3⁺ T cells are found. Of note, the hCD34⁺ hCD38⁻ CB HSC population generates myeloid- and lymphoid-restricted progenitor populations such as CMPs (common myeloid progenitors), GMPs (granulocute/monocyte progenitors), MEPs (megakaryocyte/erythrocyte progenitors) and CLPs (common lymphoid progenitors) in the bone marrow.

Isolation of human blood cells from their mouse counterparts may be performed by any method. For example, anti-human glycophorin A (anti-hGPA; Biomeda Corporation, Foster City, Calif.) antibodies which specifically recognize human erythrocytes can be used to separate human red blood cells from the mouse red blood cells and other mouse cells, by magnetic cell sorting and/or FACS. Similarly, mTerl 19 antibodies (Miltenyi Biotec Inc., Auburn, Calif.; FITC-, PE- and APC-conjugates are all available) that recognize GPA-associated protein on murine erythrocytes can be used to separate mouse red cells out from a mixture of human and mouse red blood cells, by magnetic cell sorting and/or FACS. Kina et al. The monoclonal antibody TER-119 recognizes a molecule associated with glycophorin A and specifically marks the late stages of murine erythroid lineage Br J Haematol. 2000 (109), 280-287. Human and murine platelets can also be separated immuno-magnetically, for example, using immuno-magnetic micro-beads with anti-human CD41a and/or murine CD41 antibodies.

In this manner, human hematopoeitic cells, such as erythrocytes and platelets, can be produced in a non-human mammal host and such cells will not have alpha(1,3)-galactosyltransferase-synthesized alpha-galactosyl epitopes transferred to them by the host. Optionally, the host may also be genetically modified to express or over express at least one human or non-human tolerance-promoting biomolecule as described herein, such as hDAF. The isolated human cells may, for example, be transplanted into a human patient in need thereof.

An mRNA sequence of the mouse interleukin-2 receptor gamma chain has been reported as Genbank accession no. D13821 [SEQ ID NO: 15].

Example—Tissue-Specific Deletion of Native Cells in a Xenoantigen-Reduced Non-Human Mammal Fetus to Facilitate their Replacement in a Tissue or Organ by Human Cells

A GGTA1^{-/-} (alpha(1,3)galactosyltransferase null) pig fetus that is transgenic for the liver-specific expression of a suicide gene, such as ALB-xTK that is supported by a pregnant sow, optionally GGTA1^{-/-}, is provided. For example, a GGTA1^{-/-} sow that is non-transgenic with respect to the suicide gene is bred with a GGTA1^{-/-} boar that is hemizygous or homozygous for the ALB-xTK suicide transgene. Pregnancy is confirmed with ultrasound and ganciclovir (100 mg/kg, i.v.) is administered at 40 days gestation to kill a fraction of native hepatocytes. (Generally, 40-60 days gestation is a favorable window for introducing human cells into a pig fetus). A laparotomy is performed at 45 days and each pig fetus is infused with 10×10⁶ human cord blood cells. Alternatively, human hepatocytes from isolated human cadavers can be introduced into the pig fetus(es), for example, 2 to 5 million hepatocytes injected into the liver or peritoneum of each fetal pig. Following recovery from the surgery, the prodrug may be administered on multiple occasions to the maternal host to selectively kill the fetal pig hepatocytes and thereby facilitate population of the fetal livers with human cells.

One or more rounds of prodrug administration may also be continued following birth of the animals. The resulting chimeric livers which have a substantial fraction of human cells can, for example, be explanted from the GGTA1^{-/-} pig hosts at a time following birth when the overall liver size has increased, for example, for transplantation to a human patient in need thereof.

Example—Development and Incorporation of Human Cells into Organs and Tissues of a Non-Human Mammal Fetus

A transgenic sheep fetus (or alternatively a transgenic pig fetus) is provided that expresses a suicide gene under control of a constitutive, broad-activity promoter, for example, the CMV-xTK transgene and which has reduced expression of at least one xenoantigen (with respect to human tolerance of xenogeneic material) and/or is transgenic for expression of at least one transferable tolerance-promoting biomolecule, such as hDAF and/or MIRL. The fetus is carried by a pregnant female.

Human cells capable of giving rise to hepatocytes, to other liver cells and/or to hematopoietic cells in the sheep fetus are provided for introduction into the fetus during its preimmune stage. Such human cells include but are not limited to fractionated or non-fractionated preparations of adult human bone marrow, umbilical cord blood cells, placental stem cells, and mobilized peripheral blood stem cells. Sheep fetuses are preimmune until about day 77 of gestation. The preparation and transplantation of the replacement cells may, for example, be carried out according to the method of U.S. Pub. No. 20020100065, which is incorporated by reference herein in its entirety. Human bone marrow or cord blood may, for example be fractionated, for example by magnetic cell separation and/or fluorescence activate d cell sorting, to enrich for selected phenotypes, such as those associated with hematopoietic stem cells, e.g., CD34⁺ Lin⁻ phenotypes. Unfractionated human bone marrow or human umbilical cord blood may, for example, also be used.

Preimmune fetal sheep at 55-60 days of gestation are injected with a replacement cell preparation such as 1×1 06 to 1×10⁸ (e.g., 2.5×10⁷) nucleated human cord blood cells per fetus unfractionated by phenotype, or 1-5×10⁵ CD34⁺ Lin⁻ human bone marrow cells per fetus, 1.1×10⁶ CD34⁺ Lin⁻ human cord blood cells per fetus or 1.1×10⁶ CD34⁻, Lin⁻ human cord blood cells per fetus by any suitable method, for example, intraperitoneally using a 25 gauge needle by the general technique described in Flake et al. Transplantation of fetal hematopoietic stem cells in utero: the creation of hematopoietic chimeras Science, Vol. 233, p. 766 (1986), which permits the injection of the fetus under direct visualization in an amniotic bubble through a midline laparatomy incision. Following injection of the cells, the myometrium is closed in a double layer and the pregnancy is allowed to proceed.

Previous studies in sheep show that such transplants result in significant multi-lineage human hematopoietic activity into all blood elements by about 1 month post-transplant in significant numbers of human hepatocytes at birth (about 3 months post-transplant; 5-40% of total cellularity, depending on the phenotype and dosage of the replacement cells). U.S. Pub No. 20020100065. Moreover, chimeric livers resulting from such transplantations include not only human hepatocytes that retain functional properties of normal hepatocytes, but also human endothelial and biliary duct cells, and secrete human albumin into the circulation. Almeida-Porada, Formation of human hepatocytes by human hematopoietic stem cells in sheep, Blood, (2004) 104(8) 2582-2590, which is incorporated by reference herein in its entirety. The human cells persist on a long term basis.

Blood may be collected from the chimeric animals, for example following birth, and circulating human hematopoietic cells can be isolated on the basis of human-specific antigen expression by magnetic cell sorting and/or FACS and/or, for example, where the desired cells are mitotic, by selectively killing the sheep cells using ganciclovir and/or by any method. The isolated human blood cells may be used for transplantation into a human patient in need thereof.

Chimeric solid organs and tissue may be harvested, for example, following birth, and processed as a source for human cells for transplantation into a human patient in need thereof. For example, human hepatocytes and other human cells from a chimeric liver can be isolated from the chimeric liver by reducing the chimeric liver to a cell suspension and selectively killing the non-human cells therein and/or selectively isolating the human cells from the suspension. The chimeric organs or tissues may also be used as grafts themselves for transplantation into a human patient in need thereof. In a related embodiment, a chimeric solid organ or tissue developed within the host that bears the broadly-active expression of the suicide gene (such as the chimeric liver of the example) is transplanted to a second non-human mammal that does not bear expression of the same suicide gene, where it is supported in a living state. Administration of the prodrug to the second host selectively kills the non-human cells of the chimeric organ or tissue that originate from the first non-human host mammal without harming cells of the second host, thereby promoting a more complete cellularization of the transplanted solid organ or tissue with the human cells. In this manner, a more cellularly human organ or tissue can be obtained, such as an at least substantially entirely cellularly human organ or tissue. Such an organ or tissue may, for example, be further transplanted to a human patient in need thereof.

The term “human cells” as referred in this disclosure means human cells or human-cell-derived cells. Examples include, but are not limited to, primary or cell culture passaged human cells, non-immortalized, immortalized or conditionally immortalized human cells, at least substantially human cells, genetically modified human cells, epigenetically-modified human cells, unmodified human cells, human stem cells, human progenitor cells and human differentiated cells. As described herein, the human cells may be in any form such as the whole or part of a dispersed tissue or the whole or part of a chimeric or non-chimeric, solid organ or tissue, including for example a body part. Those skilled in the art will also appreciate that non-human animals, such as non-human mammals, that are used as hosts according the invention can be genetically modified to eliminate any endogenous retroviruses that may be characteristically present in the genome of the animal. For example, swine lacking porcine endogenous retrovirus (PERV) may be used as mammal hosts and/or donors according to the invention.

As shown in this disclosure, the human cells supported by non-human animal hosts are preferably not artificially encapsulated. However, embodiments wherein the transplanted human cells supported by a non-human host are artificially encapsulated, for example, within a microporous polymeric membrane, gel or container, are also within the scope of the invention. Since host xenoantigens and tolerance-promoting biomolecules, such as hDAF, may be transferred to hosted cells via microvesicles and microparticles, such as lipoprotein particles, supporting encapsulated human cells in a xenoantigen-reduced host and/or host with expression or increased expression of a tolerance-promoting biomolecule can advantageously reduce the transfer of xenoantigens to the hosted cells and/or cause or increase the transfer of tolerance-promoting biomolecules to the hosted cells, respectively.

The term “promoter” as used herein should be construed broadly, for example, as including promoters and enhancers and combinations thereof. The term “expression” as used herein with respect to a carbohydrate epitope xenoantigen relates to the amount of presentation of the epitope in its xenoantigenic state. Accordingly, as described herein, reducing the expression of a carbohydrate epitope xenoantigen may, for example, be accomplished by reducing or eliminating the activity of one or more enzymes that produce the carbohydrate epitope xenoantigen, by providing enzyme activities that compete with substrate for such carbohydrate xenoantigen-producing enzymes, by enzymatically cleaving the carbohydrate epitope xenoantigen and/or by otherwise modifying the carbohydrate xenoantigen to a less xenoantigenic structure or state.

Modifications of a host that reduce or completely eliminate the expression of a xenoantigen may be of any sort, such as genetic modifications or epigenetic modifications, and may be introduced in any current or prior generation so long as the host comprises the modification(s). Genetic modifications that inactivate a gene or an allele of a gene may be of any sort and may, for example, include mutations of the promoter of a gene that reduce or eliminate transcription of the gene and/or mutations in the normally transcribed sequence of gene that prevent expression of the transcript (such as elimination of a necessary start codon or ribosome-binding sequence) or prevent expression of a functional protein product. Genetic mutations of a gene sequence may be of any sort, such as deletions, insertions, substitutions, inversions and/or combinations thereof. Another kind of genetic modification of a host that can reduce the expression of a xenoantigen involves integration of a transgene into the host (in any current or prior generation), wherein the transgene produces a gene product, such as an RNA or protein, that has the effect of reducing the expression of the xenoantigen. In one example, a genomically integrated transgene that drives the expression (e.g., constitutive or inducible) of an RNA-silencing molecule that silences the mRNA transcripts of a selected gene, such as a gene for a protein xenoantigen or for a xenoantigen-producing enzyme, is used. In another example, a genomically integrated transgene that drives the expression (e.g., constitutive or inducible) of a gene that produces a gene product that cleaves or alters a xenoantigen is used. In still another example, a genomically integrated transgene that drives the expression (e.g., constitutive or inducible) of a gene that produces an enzyme that competes for substrate with a xenoantigen-producing enzyme can be used. Epigenetic modifications can also reduce or completely eliminate the activity of a gene. For example, double-stranded RNA-mediated gene silencing of a promoter of a gene and/or the other parts of the gene can silence transcription of the gene. While not being limited by theory, RNA-mediated gene silencing is believed to be mediated by methylation and/or other modifications of a gene at the DNA level.

Issued United States Patents are identified herein with the prefix “U.S.” followed by the patent number. Published United States Patent Applications are identified herein with the prefix “U.S. Pub. No.” followed by the publication number. Each of the patents, patent applications, genetic sequences, articles and other publications cited in this disclosure is incorporated by reference in its entirety as if each was set forth herein.

The following U.S. Patents, which may or may not be cited elsewhere in this disclosure, are incorporated by reference herein in there entireties:

U.S. Pat. No. 6,923,959; U.S. Pat. No. 6,916,654; U.S. Pat. No. 6,911,220; U.S. Pat. No. 6,825,395; U.S. Pat. No. 6,794,182; U.S. Pat. No. 6,758,865; U.S. Pat. No. 6,734,295; U.S. Pat. No. 6,718,986; U.S. Pat. No. 6,700,037; U.S. Pat. No. 6,699,231; U.S. Pat. No. 6,689,598; U.S. Pat. No. 6,680,305; U.S. Pat. No. 6,677,150; U.S. Pat. No. 6,673,987; U.S. Pat. No. 6,673,594; U.S. Pat. No. 6,660,905; U.S. Pat. No. 6,558,663; U.S. Pat. No. 6,498,285; U.S. Pat. No. 6,649,595; U.S. Pat. No. 6,642,045; U.S. Pat. No. 6,639,122; U.S. Pat. No. 6,638,747; U.S. Pat. No. 6,627,393; U.S. Pat. No. 6,589,223; U.S. Pat. No. 6,582,953; U.S. Pat. No. 6,576,464; U.S. Pat. No. 6,562,619; U.S. Pat. No. 6,524,785; U.S. Pat. No. 6,521,448; U.S. Pat. No. 6,514,752; U.S. Pat. No. 6,506,549; U.S. Pat. No. 6,500,929; U.S. Pat. No. 6,495,735; U.S. Pat. No. 6,492,103; U.S. Pat. No. 6,491,912; U.S. Pat. No. 6,469,229; U.S. Pat. No. 6,447,767; U.S. Pat. No. 6,423,316; U.S. Pat. No. 6,368,572; U.S. Pat. No. 6,413,769; U.S. Pat. No. 6,368,572; U.S. Pat. No. 6,353,150; U.S. Pat. No. 6,342,344; U.S. Pat. No. 6,331,658; U.S. Pat. No. 6,331,319; U.S. Pat. No. 6,325,999; U.S. Pat. No. 6,296,846; U.S. Pat. No. 6,280,718; U.S. Pat. No. 6,258,998; U.S. Pat. No. 6,255,474; U.S. Pat. No. 6,252,136; U.S. Pat. No. 6,245,566; U.S. Pat. No. 6,215,039; U.S. Pat. No. 6,194,635; U.S. Pat. No. 6,187,757; U.S. Pat. No. 6,172,190; U.S. Pat. No. 6,166,288; U.S. Pat. No. 6,136,954; U.S. Pat. No. 6,132,708; U.S. Pat. No. 6,129,911; U.S. Pat. No. 6,100,443; U.S. Pat. No. 6,100,082; U.S. Pat. No. 6,093,872; U.S. Pat. No. 6,090,622; U.S. Pat. No. 6,060,049; U.S. Pat. No. 6,046,379; U.S. Pat. No. 6,046,046; U.S. Pat. No. 6,030,833; U.S. Pat. No. 6,018,096; U.S. Pat. No. 5,976,524; U.S. Pat. No. 5,965,433; U.S. Pat. No. 5,925,802; U.S. Pat. No. 5,912,411; U.S. Pat. No. 5,871,997; U.S. Pat. No. 5,866,757; U.S. Pat. No. 5,849,991; U.S. Pat. No. 5,821,117; U.S. Pat. No. 5,807,737; U.S. Pat. No. 5,752,929; U.S. Pat. No. 5,709,843; U.S. Pat. No. 5,672,346; U.S. Pat. No. 5,652,373; U.S. Pat. No. 5,643,746; U.S. Pat. No. 5,639,939; U.S. Pat. No. 5,633,426; U.S. Pat. No. 5,625,127; U.S. Pat. No. 5,599,659; U.S. Pat. No. 5,589,362; U.S. Pat. No. 5,586,438; U.S. Pat. No. 5,556,954; U.S. Pat. No. 5,523,226; U.S. Pat. No. 5,498,427; U.S. Pat. No. 5,494,822; U.S. Pat. No. 5,476,997; U.S. Pat. No. 5,418,147; U.S. Pat. No. 5,416,260; U.S. Pat. No. 5,362,622; U.S. Pat. No. 5,326,706; U.S. Pat. No. 4,837,390; U.S. Pat. No. 4,745,759; and U.S. Pat. No. 4,186,565.

The following Published Patent Applications, which may or may not be cited elsewhere in this disclosure, are incorporated by reference herein in there entireties:

U.S. Pub. Nos. 20050201990 (Ser. No. 11/076,668); 20050177883 (Ser. No. 10/470,785); 20050176139 (Ser. No. 11/032,153); 20050170452 (Ser. No. 10/500,240); U.S. Pub. No. 20050164210 (Ser. No. 10/763,479); 20050155095 (Ser. No. 10/981,935); 20050155094(Ser. No. 10/503,464); 20050148072 (Ser. No. 10/944,919); 20050142121 (Ser. No. 10/949,411); 20050125853 (Ser. No. 10/505,760); 20050120400 (Ser. No. 10/499,407); 20050112122 (Ser. No. 10/933,933); 20050108783 (Ser. No. 10/947,920); 20050076399 (Ser. No. 10/500,748); 20050028230 (Ser. No. 10/843,038); 20040268424 (Ser. No. 10/646,970); 20040258669 (Ser. No. 10/701,789); 20040209357 (Ser. No. 10/769,686); 20040191228 (Ser. No. 10/487,944); 20040180041 (Ser. No. 10/809,556); 20040171824 (Ser. No. 10/469,881); 20040171155 (Ser. No. 10/762,888); 20040171138 (Ser. No. 10/640,867); 20040136972 (Ser. No. 10/759,033); 20040110286 (Ser. No. 10/313,195); 20040073963 (Ser. No. 10/362,429); 20030224350 (Ser. No. 10/113,664); 20030211098(Ser. No. 10/181,896); 20030206891 (Ser. No. 10/341,967); 20030203427 (Ser. No. 10/125,994); 20030165480 (Ser. No. 10/135,919); 20030147859 (Ser. No. 09/881,721); 20030131365 (Ser. No. 10/172,459); 20030115616 (Ser. No. 10/243,087); 20030096410 (Ser. No. 09/178036); 20030092174 (Ser. No. 10/147,286); 20030086909 (Ser. No. 09/222,460); 20030068818 (Ser. No. 10/105,963); 20030068818 (Ser. No. 10/105,963); 20030049235 (Ser. No. 09/477,737); 20030014770 (Ser. No. 10/098,276); 20030068308 (Ser. No. 10/132,443); 20030003574 (Ser. No. 10/099,539); 20030003083 (Ser. No. 10/169,028); 20030198628 (Ser. No. 10/395,552); 20020197240 (Ser. No. 10/146,092); 20020187972 (Ser. No. 09/949,278); 20020164571 (Ser. No. 09/798,790); 20020100065 (Ser. No. 09/895,895); 20020090370 (Ser. No. 09/753,007); 20020031494 (Ser. No. 10/254,077); 20010053362 (Ser. No. 09/802,350); and 20010049139 (Ser. No. 08/816,750).

In addition, the following published international applications and their related U.S. applications are each incorporated by reference in their entireties: International Pub. No. No. WO 2004/108904 A2 of PCT/US2004/018106 and U.S. Prov. Ser. No. 60/476,396 to which priority is claimed; International Pub. No. WO 2004/027029 A2 of PCT/US2003/029251, U.S. Prov. Ser. No. 60/411,790 to which priority is claimed, and the U.S. national phase Ser. No. 10/527,587; and International Pub. No. WO 2004/016742 A2 of PCT/US2003/025199 and U.S. Prov. Ser. No. 60/403,405 to which priority is claimed.

It should be understood that the embodiments and examples set forth within this disclosure are meant to illustrate various aspects of the invention and are not limiting of its scope. Many embodiments and variations within the spirit and scope of the invention may be apparent to those of skill in the art upon reviewing this disclosure.

What is claimed is:

1. A method for supporting human cells in a non-human, mammalian host animal, comprising the steps of:
transplanting human cells to a non-human, mammal host,
wherein the host is at least substantially immunologically tolerant of the transplanted human cells,
wherein the human cells are supported in a living state by the host, and
wherein the host is modified or treated to reduce the expression of at least one xenoantigen defined with respect to a normal human immune system.

2. The method of claim 1, wherein the at least one xenoantigen comprises Gal.alpha.(1,3)Gal epitopes.

3. The method of claim 1, wherein the host comprises at least one modification that reduces the expression of at least one xenoantigen defined with respect to a normal human immune system.

4. The method of claim 1, wherein the host comprises at least one genetic modification that reduces the expression of Gal.alpha.(1,3)Gal epitopes.

5. The method of claim 1, further comprising the step of: treating the host to reduce expression by the host of at least one xenoantigen defined with respect to a normal human immunological system.

6. The method of claim 1, wherein the host further comprises at least one genetic modification that causes or increases the expression of at least one preselected tolerance-promoting biomolecule.

7. The method of claim 1, further comprising the step of selectively killing at least some of the native cells of the host.

8. The method of claim 1, further comprising the step of:
after a period of time of support by the host, removing at least some human cells supported by the host from the host.

9. The method of claim 1, wherein the step of transplanting human cells to a non-human, mammal host comprises:
transplanting non-encapsulated human cells to the non-human, mammal host.

10. A non-human animal, comprising:
a non-human mammal host; and
human cells supported in a living state by the host,
wherein, the host is at least substantially immunologically tolerant of the human cells, and
wherein, the host:
(i) comprises at least one modification that reduces the expression of at least one xenoantigen defined with respect to a normal human immune system, or
(ii) has reduced expression of at least one xenoantigen defined with respect to a normal human immune system as a result of treatment of the host, or
(iii) both (i) and (ii).

11. The animal of claim 10, wherein the human cells comprise non-encapsulated human cells.

12. The animal of claim 10, wherein the host comprises at least one modification that reduces the expression of at least one xenoantigen defined with respect to a normal human immune system.

13. The animal of claim 10, wherein the at least one xenoantigen comprises Gal.alpha.(1,3)Gal epitopes.

14. The animal of claim 10, wherein the host comprises at least one genetic modification that reduces the expression of at least one xenoantigen defined with respect to a normal human immune system.

15. The animal of claim 10, wherein the at least one xenoantigen comprises Gal.alpha.(1,3)Gal epitopes.

16. The animal of claim 10, wherein the host has reduced expression of at least one xenoantigen defined with respect to a normal human immune system as a result of treatment of the host.

17. The animal of claim 10, wherein the at least one xenoantigen comprises Gal.alpha.(1,3)Gal epitopes.

18. The animal of claim 10, wherein the host further comprises at least one genetic modification rendering at least some of the native host cells selectively and conditionally killable versus the human cells.

19. The animal of claim 10, wherein the host further comprises at least one genetic modification causing or increasing expression of at least one tolerance-promoting biomolecule.

20. The animal of claim 10, wherein the human cells:
(i) comprise human hematopoietic cells;
(ii) comprise human hematopoietic progenitor cells that produce differentiated human hematopoietic cells within the host;
(iii) comprise human erythrocytes produced within the host;
(iv) comprise human platelets produced within the host;
(v) comprise human cells integrated into at least one solid or dispersed organ or tissue of the host;
(vi) comprise human cells integrated into a chimeric solid organ or chimeric solid tissue supported by the host, the organ or tissue comprising human cells and non-human mammal cells;
(vii) comprise human cells of at least part of a functionally developed solid human organ or functionally developed solid human tissue supported by the host;
(viii) comprise human cells of at least part of a human organ anlagen or human tissue anlagen supported by the host;
(ix) comprise human cells of a part of a human body supported by the host; or
(x) any combination of (i)-(ix).


SEQUENCE LISTING (15 sequences)

SEQ ID NO: 1 - DNA, 224 bp, Sus scrofa
gaattgaccaggtcttgtggagaaaacagatccagacggcaaacatacgcaagggatttagtcaaacacatttttggcaaaaaaactatgaattttgtaatcagttgtgagccaatgaaatacaaaaatgagtctagttaataatctacaattattggttaaagaagtatattagtgctgactttcctctgttcgtcctaccttttcttttctatcaaccccac

SEQ ID NO: 2 - DNA, 2241 bp, Artificial
  Chimeric transgene construct comprising porcine albumin promoter/enhancer operably linked to mutant Herpes virus thymidine kinase (xTK) gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactgcaggaattgaccaggtcttgtggagaaaacagatccagacggcaaacatacgcaagggatttagtcaaacacatttttggcaaaaaaactatgaattttgtaatcagttgtgagccaatgaaatacaaaaatgagtctagttaataatctacaattattggttaaagaagtatattagtgctgactttcctctgttcgtcctaccttttcttttctatcaaccccacatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaactgcccacgctactgcgggtttatatagacggtctccacgggctggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagaattcgctagctctctagtcgagaattcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctaagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggcctt

SEQ ID NO: 3 - DNA, 1506 bp, Artificial
  Chimeric transgene construct comprising porcine alpha-fetoprotein promoter/enhancer operably linked to fungal cytosine deaminase gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactgcagtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacaccatggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtaggctagctctctagtcgagaattcgctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaat

SEQ ID NO: 4 - DNA, 2294 bp, Artificial
  Chimeric transgene construct comprising cytomegalovirus promoter operably linked to mutant Herpes virus thymidine kinase (xTK) gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagataatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggcctt

SEQ ID NO: 5 - DNA, 1782 bp, Artificial
  Chimeric transgene construct comprising cytomegalovirus promoter operably linked to fungal cytosine deaminase gene
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatcctgcagatggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtaggctagctctctagtcgagtccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattcttattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 1269 bp, Sus scrofa
  CDS (16)..(1128)
catgaggagaaaataatgaatgtcaaaggaagagtggttctgtcaatgctgcttgtctcaactgtaatggttgtgttttgggaatacatcaacagcccagaaggttctttgttctggatataccagtcaaaaaacccagaagttggcagcagtgctcagaggggctggtggtttccgagctggtttaacaatgggactcacagttaccacgaagaagaagacgctataggcaacgaaaaggaacaaagaaaagaagacaacagaggagagcttccgctagtggactggtttaatcctgagaaacgcccagaggtcgtgaccataaccagatggaaggctccagtggtatgggaaggcacttacaacagagccgtcttagataattattatgccaaacagaaaattaccgtgggcttgacggtttttgctgtcggaagatacattgagcattacttggaggagttcttaatatctgcaaatacatacttcatggttggccacaaagtcatcttttacatcatggtggatgatatctccaggatgcctttgatagagctgggtcctctgcgttcctttaaagtgtttgagatcaagtccgagaagaggtggcaagacatcagcatgatgcgcatgaagaccatcggggagcacatcctggcccacatccagcacgaggtggacttcctcttctgcatggacgtggatcaggtcttccaaaacaactttggggtggagaccctgggccagtcggtggctcagctacaggcctggtggtacaaggcacatcctgacgagttcacctacgagaggcggaaggagtccgcagcctacattccgtttggccagggggatttttattaccacgcagccatttttgggggaacacccactcaggttctaaacatcactcaggagtgcttcaagggaatcctccaggacaaggaaaatgacatagaagccgagtggcatgatgaaagccatctaaacaagtatttccttctcaacaaacccactaaaatcttatccccagaatactgctgggattatcatataggcatgtctgtggatattaggattgtcaagatagcttggcagaaaaaagagtataatttggttagaaataacatctgactttaaattgtgccagcagttttctgaatttgaaagagtattactctggctacttcctcagagaagtagcacttaattttaacttttaaaaaaatactaacaaaataccaacacagtaagtacatattattcttcctt

SEQ ID NO: 7 - DNA, 1121 bp, Ovis aries
  CDS (11)..(1120)
ggagaaaataatgaatgtcaaaggaaaagtgattctgtcaatgctggttgtctcaactgtcattgttgtgttttgggaatatatccacagcccagaaggctctttgttctggataaacccatcaagaaacccagaagtcagtggcggcagcagcattcagaagggctggtggtttccgagatggtttaacaatggttaccaagaagaagatgaagacgtagacgaagaaaaggaacaaagaaaggaagacaaaagcaagcttaagctatcggactggttcaacccatttaaacgccctgaggttgtgactatgacagattggaaggcacccgtggtgtgggaaggcacttacaacagagccgtcttagacgattactacgccaagcagaaaattaccgtcggcctgacggttttcgccgtcggaagatacattgagcattacttggaggagttcttaacgtctgctaataagcacttcatggttggccaccgagtcatcttttacgtcatggtggacgatgtctccaggatgcctttgatagagctgggccctctgcgctccttcaaagtgtttgaggtcaagcctgagaggaggtggcaggacgtcagcatggtgcgcatgaagaccatcggggagcacatcgtggcccacatccaacgtgaggttgacttcctcttctgcatggacgtggaccaggtcttccaagatgagttcggggtggagaccctgggtgagtcggtggcccagctacaggcctggtggtacaaggcagatcccgatgagtttacctacgagaggcgcaaggagtctgcagcatatattcccttcggcgaaggggatttttattaccacgcagccatttttgggggaacacccactcaggtccttaacatcacccaggaatgcttcaaaggaatcctcaaggacaagaaaaatgacatagaagcccaatggcatgatgagagccatctaaacaagtatttccttctcaacaaacccactaaaatcttatccccggaatactgctgggattatcatataggcctacctgcggatattaagcttgtcaagatgtcttggcagacaaaagagtataatttggttagaaataatgtctgac

SEQ ID NO: 8 - DNA, 1655 bp, Bos taurus
  CDS (469)..(1575)
ccgggggccgggccgagctgggagcgtcgagcccgctgcccagcgcccgccggctccctcgcgcccctgcccgccgccccggaggagcgcccggcggccggccgacgggagcgcagcggcacaccccgccccggcacgcccgcggggctcgggaggaggcagcgcgccgactgttccggcagccgaggacgccgccggggagccgaggcgccggccagcccccagcgcgcccagcttctgcggatcagggaaaccacgtgtcctcaagtggccagccagctgtccccaagaggaacttgcctggcatttgcacggaaagacgagacacttcacaaaatcaacggagtcagaaggctgcaccttcgcttcctcccagccctgcctccttctgcagaacggagctcagtagaacttggtacttttgccttttactctaggaggagagaagcagacgatgaggagaaaataatgaatgtcaaaggaaaagtgattctgtcaatgctggttgtctcaactgtcattgttgtgttttgggaatatatccacagcccagaaggctctttgttctggataaacccatcaagaaacccagaagttggtggcagcagcattcagaagggctggtggcttccgagatggtttaacaatggttaccatgaagaagatggagacataaacgaagaaaaggaacaaagaaacgaagacgaaagcaagcttaagctatcggactggttcaacccatttaaacgccccgaggttgtgaccatgacgaagtggaaggctccagtggtgtgggaaggcacttacaacagagccgtcttagacaattattatgccaagcagaaaattaccgtcggcctgacggttttcgccgtcggaagatacattgagcattacttggaggagttcttaacgtctgctaataagcacttcatggtgggccacccagtcatcttttatatcatggtagatgatgtctccaggatgcctttgatagagttgggtcctctgcgctccttcaaagtgtttaagatcaagcctgagaagaggtggcaggacatcagcatgatgcgcatgaagactatcggggagcacattgtggcccacatccagcatgaggttgacttccttttctgcatggatgtggaccaggtcttccaagacaagtttggggtggagaccctgggcgagtcggtggcccagctacaagcctggtggtacaaggcagatcccaatgacttcacctacgagaggcggaaggagtctgcagcatacattcccttcggcgaaggggatttttattaccatgcagccatttttgggggaacacccactcaggtccttaacatcacccaggaatgcttcaaaggaatcctcaaggacaagaaaaatgacatagaagcccaatggcatgatgaaagccatctaaacaagtatttccttctcaacaaacctactaaaatcttatccccggaatactgctgggattatcacataggcctacctgcggatattaagcttgtcaagatgtcttggcagacaaaagagtataatgtggttagaaataatgtctgactttgtgccagtacatttctgaatttgagagagtattattctggctacttcctcagaaaagtaacacttaattttaactt

SEQ ID NO: 9 - DNA, 3450 bp, Mus musculus
  CDS (445)..(1560)
cgtcttaggaggctggagattctgggtggagccctagccctgccttttcttagctggctgacaccttcccttgtagactcttcttggaatgagaagtaccgattctgctgaagacctcgcgctctcaggctctgggagttggaaccctgtaccttcctttcctctgctgagccctgcctccttcggcaggccagagctcgacagaagctcggttgctttgctgtttgctttggagggaacacagctgacgatgaggctgactttgaactcaagagatctgcttaccccagtctcctggaattaaaggcctgtactaccttgcctggacctaagattttcatgatcactatgcttcaagatctccatgtcaacaagatctccatgtcaagatccaagtcagaaacaagtcttccatcctcaagatctggatcacaggagaaaataatgaatgtcaagggaaaagtaatcctgttgatgctgattgtctcaaccgtggttgtcgtgttttgggaatatgtcaacagcccagacggctctttcttgtggatatatcacacaaaaattccagaggttggtgagaacagatggcagaaggactggtggttcccaagctggtttaaaaatgggacccacagttatcaagaagacaacgtagaaggacggagagaaaagggtagaaatggagatcgcattgaagagcctcagctatgggactggttcaatccaaagaaccgcccggatgttttgacagtgaccccgtggaaggcgccgattgtgtgggaaggcacttatgacacagctctgctggaaaagtactacgccacacagaaactcactgtggggctgacagtgtttgctgtgggaaagtacattgagcattacttagaagactttctggagtctgctgacatgtacttcatggttggccatcgggtcatattttacgtcatgatagatgacacctcccggatgcctgtcgtgcacctgaaccctctacattccttacaagtctttgagatcaggtctgagaagaggtggcaggatatcagcatgatgcgcatgaagaccattggggagcacatcctggcccacatccagcacgaggtcgacttcctcttctgcatggacgtggatcaagtctttcaagacaacttcggggtggaaactctgggccagctggtagcacagctccaggcctggtggtacaaggccagtcccgagaagttcacctatgagaggcgggaactgtcggccgcgtacattccattcggagagggggatttttactaccacgcggccatttttggaggaacgcctactcacattctcaacctcaccagggagtgctttaaggggatcctccaggacaagaaacatgacatagaagcccagtggcatgatgagagccacctcaacaaatacttccttttcaacaaacccactaaaatcctatctccagagtattgctgggactatcagataggcctgccttcagatattaaaagtgtcaaggtagcttggcagacaaaagagtataatttggttagaaataatgtctgacttcaaattgtgatggaaacttgacactattactctggctaattcctcaaacaagtagcaacacttgatttcaacttttaaaagaaacaatcaaaaccaaaacccactaccatggcaaacagatgatttctcctgacaccttgagcctgtaatatgtgagaaagagtctatggcaagtaatcaggtataaattctcaatgatttcttatatattctgggtcttgggaaaacttgattctagaaatcaaaattaatttgacaaaggaaaagcagatgccggaaacttcttcccagtctgtcatacaattcaccactggccaggtgctgagagaagcattagggaacagtgtgggttgtgtcagagttggacggctccatccctttggcttcattatcttcctcctcatggagattctaaagcaacccagagaggctttgcagccagagacctttaataaggatgccaatgtgaccatcagtctgtaaaagctgatggctccaggagcgctggcagtccaggccccactaggctattgtttctgtcctgggcataaaggaggcagagagtgccaataggtactttggtggcacatgttcagagtccaggaaaaatcaagggtgaccacttagagggacataggacttggggttggtgattgaactgagttacaaacacagacagctttcttcaggatgactaacagcaggaattgaatggaaagtgtgttcattttgttttgcccaaattgtattcatgctgttagctttgtgtgttgagccctgtggagagggtgtgactgtatcagggaaggagagtacctcagcggactgaggaccagcaccctattatatcagaagacaatctctcatcatcaggtcctacctacaacctgctctgaacctccgagttcctcagcccatcgtgttccagtgtgggggcctgtatggagcaggtgactgaagacaaagccccctgtcacatgacctcatttcccctgctctagtactatgcaagtgtgacagccagccagccagatgtactggacaacataggaaccgactttatggcaatgggagccgcagtcactacaacggagctgctgaaggttctgttccccgctctgagagcctgcaggagcccctgtataggtggttctcaacctatgggtcgcgacccctttgggaagtgttaaatgaccctttcacaggtgtcccctaagacggttaaaaaacatagatatttccactctgactggtaacagtagcagaattacagttatgaaatagcaagggaaataattctggggttcgtgtcatccataccatgaggagctacattaggtcacatcattagggaagttgagaagcatagctctacttgggtatttaagcaaattatgcaaagggggttgtcgctctgtgttctgtgtatgcatatatttatattttgcttgtcttccagtttaggtcaatctgtttcttcctttaagcagtttatttaaaaggccattgcaccatcttggtgaacagcatgaggggtttcaataaaaaataggatcttacctttgtccacagggctctacctcttacttttcaattgtgaacaaaaaaggtcgcacacccagaggcaacaaaacccacagaattcctgaaccaatgggagatgccaatggaagcagagcttgcacatctgctaaaaattctgcctctctgtcactgtgctggatccgtctaaagtgggacagttcaatggtctgaaagtttcaaaaaggctggggaatttgaggggatttttttttaaaataaaattgatccaagtttaaatctctaatgagtaagcttaggattttattaaaggtaatttttagacattcttcaaaataagaattc

SEQ ID NO: 10 - DNA, 1936 bp, Rattus norvegicus
  CDS (78)..(1097)
tttcgagcaggtcatttcttctatctctctgggctggaaaaaactgcatggcaggttttcaccactgacttctccccatggctctggagggactcagggccaagaagagactcctgtggcgactcttcctgtctgcgtttggtctcctaggcctgtaccattactggttcaagattttcaggctctttgaagtgttcatccccatgggcatctgccctatggccatcatgcctctgctaaaggacaatttcaccggagtgctgcgtcactgggcccggcctgaagtcctgacctgtacctcttggggagccccaattatctgggatgagaccttcgacccacacgtagctgagcgagaggcgagacggcagaaccttaccattggactgactgtctttgcggtaggcaggtacctggagaagtacctggagcacttcctggtatcggcagagcagtacttcatggtcggccagaacgtggtgtattatgtgtttaccgaccgaccggaagcagtgccccacgtggctctaggccagggtcgcctgctgcgggtgaaaccagtgagaagagagaagcgctggcaggacgtgtcaatggctcgcatgctcacgctacacgaggctctgggagggcagctgggccgagaagctgactatgtgttctgcctggacgtggaccagtacttcagcggtaacttcggacccgaggtgctggcagatttggtggcacagctgcacgcctggcacttccgctggcctcggtggatgctgccctacgagagggacaagcggtcggctgccgcgctgtcgttaagcgaaggcgatttctactaccacgccgcggtgttcgggggcagtgtggcggctctgctcaagctgacggctcactgtgcgactggccaacagctggaccgtgagcatggcattgaggcacgctggcacgacgagagccacctcaacaagttcttctggctgagcaagcccaccaagctgctgtcgccagagttttgctgggcagaggaaattggctggaggccagagatccaccaccctcgtctgatctgggcacccaaggagtacgcactggtgcgaacctagcaggccaccctggccaggcgggcacaggaacgtctttggcactgcgagtgttgaatggaattgcaaaatgtttcaaaagcacgaagtcagagccagttacactgcctttggaagggggacttcggaactcagaggtactgtgcctggggccgtacacccttgtcagctggccagagagcctttttgctccaaccagatgcaaattatggaaaggcaagagtttgtcagcttactttgcctacaaggccttccttagcccttggtaatggttggtgtctcagcttctgtccggaccttaggtttgacagctgtctggagccattcctgtgaccaggacctgcagattctcttctcttgctcttcacggtcttctgttctacttcagcccccagctctccattctctgcctgtgctaacatgcagctccctgtctgcatttgttcaactgcttcttcctccgtcatcaggaaggtttccattcctgcagctgggctggcttcctgacccaatgagcacttccccttcagctcccaagcagctggctgccttgctgctgttgtagtgtggctggcagaccactcggttgtcacggcctctgccaccaccacggctgtctcttcatgttcctagacagccctcagccttggcaaggccattcaagggaagcctcttgtgggacccttcagctcggtcggtgtcatgtaggagcacgggagagtgttggctctcctccccaaccccatccctgcatgctagctcactaagggcagctgatcatctagttttacagccataaaagaggtgtttaagccaaaaaaaaaaaaaaaaaaaaaaaaaaa

SEQ ID NO: 11 - DNA, 1113 bp, Mus musculus
  CDS (1)..(1113)
atggctctggggacagagttgggagtgagctggccagggtcacatggaagttgccgagaacaagaaggacagagacaaagaggcccagggaagccaacctggggactttcacgggccaagaagagactcctgtggcggttcttcctgtctgcatttggtttcttaggcctgtaccattacaggttcattattatcaggctcatagaaggctccatccccatgggcacctgccctacagccataatgcctctgccgagggacaatttcacaggagtactgcaccactgggcccggcctgaagtcctgacctgtacctcttggggagcaccaattatttgggatggcactttcgaccctcatgtagcccagcaagaggcgagacggcggaacctcaccatcgggctgactgtgtttgctgtaggcaggtacctggagaagtacctggaacacttcctggtatcggcagagcagcacttcatggtcggccagaacgtggtgtactatgtgtttacggatcgcccggaagcagtgccctatgtggctctaggccagggtcgcctgctgcgggcaaaacccgtgcagcgagagaggcgctggcaggacgtgtccatggcacgcatgcccacgctacacgaggctctgggagggcagctgggccaagaagctgactttgtgttctgcctggacgtggaccagtacttcaccggtaacttcgggcctgaggtgctggcagatttggtggcacagctgcacgcctggcactaccgctggccgcggtggctgctgccctacgagagggacaagcgatcggctgctgcgctgtcgttaagcgaaggcgatttctactaccacgctgcggtgtttggcggcagtgtggctgcactgctcaaactgacggcccactgtgcgactggccaacagctggaccataagcgcggcattgaggcactctggcacgacgaaagccaccttaacaagttcttctggctgaacaagcccaccaagctgctgtcgcctgagttctgctgggcagaggaaattatctggaggagagagatccatcacccacgcctgctctgggcacccaaggaatatacgctggtgcgaaactag

SEQ ID NO: 12 - DNA, 672 bp, Sus sp.
  CDS (1)..(672)
ctagttgtagaaaaggatgaagaaaatggagttttgcttctagaactaaatcctcctaacccgtgggattcagaacccagatctcctgaagatttggcttttggggaagtgcagatcacgtaccttactcacgcctgcatggacctcaagctgggagacaagaggatggtgttcgatccttggttaatcggtcctgcttttgcgcgaggatggtggttactacacgagcctccatctgattggctggagaggctgagccgcgcagatttaatttacatcagtcacatgcactcagaccacctgagttacccaacactgaagaagcttgctgagagaagaccagatgttcccatttatgttggcaacacggaaagacctgtattttggaatctgaatcagagtggcgtccagttgactaatatcaatgtagtgccatttggaatatggcagcaggtagacaaaaatcttcgattcatgatcttgatggatggcgttcatcctgagatggacacttgcattattgtggaatacaaaggtcataaaatactcaatacagtggattgcaccagacccaatggaggaaggctgcctatgaaggttgcattaatgatgagtgattttgctggaggagcttcaggctttccaatgactttcagtggtggaaaatttact

SEQ ID NO: 13 - DNA, 2282 bp, Mus musculus
  CDS (461)..(2191)
ggaccctccattctggcagtggcagctgtgaagtctagcgatcttcttctggagagaacagatcagcagggtggagctgggagaggggtagtgttgatgttagacacataatgtgacatcagcctgctctgcatctgtgtctccagaggaacttgaacttcccagctgtactttggtaattgcttctgaaaaaaacctcctggccacacagaaatcaaataaaatttttaatcaagcaaattgaaaaatccaggctgtattataaacatcaattaaaagaagatctgggcctgggccttttgatgtttcctgaccagcttggatgctgtcacctttgtctactcagaagggagtaaaggcagtctaaagtcatcctcgccatcctcgccttcctggtgtgattcgcagaagtagaaagaacaccagcagctgctttgaaataccctggagctggcagatgatggacaggaaacagacagctgagaccctgctgaccctgtctcctgctgaagttgccaacctcaaggaagggatcaatttttttcgaaataagactactgggaaagagtacattttatacaaggagaaggaccatctaaaggcatgcaagaacctctgcaagcaccagggaggcctgttcatgaaagacatcgaggatttagatggaaggtccgttaaatgcacaaagcacaactggaagttagacgtgagcaccatgaaatatatcaaccctccagggagcttctgtcaagacgagctcgttattgaaatggatgaaaacaatgggctttccctggtagaactgaaccctcctaacccctgggactctgatcccaggtctcctgaagaattagcttttggggaagtacagataacatatctcactcatgcctgcatggacctcaagttgggagacaagcgaatggtatttgacccttggttaattggccctgcttttgcccgaggatggtggttgctacatgagcctccatctgactggttggagaggctgtgcaaagcagacctcatttatatcagccacatgcactcagaccacctgagctaccctaccctgaagcagctttcccagagacgaccagacattcccatttatgttggcgacacagaaaggcctgtgttttggaacctggatcagagtggcgtcgggttaactaacatcaacgtggttccatttggaatatggcaacaggtagacaaaagtctgcggttcatgatcttgatggacggcgttcatcctgagatggacacatgcattatcgtggagtacaaaggtcataaaatactcaacacagtggactgcaccagacccaatgggggaaggcttcctgagaaagttgctctaatgatgagtgatttcgcaggaggtgcatcaggctttccaatgactttcagtggtggaaaatttactgaggaatggaaagcccagttcattaaggctgaaagaagaaagcttctgaattacaaagctcagctggtgaaggacctgcagccccgaatctactgtccgtttgctgggtactttgtggagtctcacccatctgacaagtacattaaggaaacaaacaccaaaaatgacccaaatcagctcaacaatcttatcaggaaaaactctgacgtggtgacatggaccccacgacctggcgctgtcctcgaccttggcaggatgctgaaggacccaacagacagcaagggcattgtggagcctccagaggggacaaagatttacaaggattcctgggactttggcccgtacctggagatcttgaattctgctgtcagagatgaaatcttctgtcattcatcctggattaaagagtacttcacgtgggctggatttaagaattacaacctggtggtcaggatgattgaaacagatgaagatttcagcccttttcctggagggtacgactatctggtggactttctagatttatcctttccgaaagaaagacccagccgggagcatccttatgaagaaatccatagccgggtggatgtcatcaggtacgtggtgaagaacggcctgctgtgggatgatctgtatattggattccagacccgattgctgcgggaccctgatatataccatcatctgttttggaatcattttcagataaaactccctctaacaccacccaactggaagtcgttcctaatgcactgtgattagtctggacctggtaagtcccaggaccccagcccagaggatggtgcctgaacattcaagatgggtctcccctgcttcgataaacctttctgga

SEQ ID NO: 14 - DNA, 2444 bp, Mus musculus
  CDS (623)..(2353)
ggaccctccattctggcagtggcagctgtgaagtctagcgatcttcttctggagagaacagatcagcaggcaataatggagtgttcccggtcaactctcatggcctcaggactctcacagccagcagtttccagatatggggattgatccccttcagtgcctcttgggacatggaatattggcagactctctctggatctgctcctggattgagctctacaaatacaggagggtggagctgggagaggggtagtgttgatgttagacacataatgtgacatcagcctgctctgcatctgtgtctccagaggaacttgaacttcccagctgtactttggtaattgcttctgaaaaaaacctcctggccacacagaaatcaaataaaatttttaatcaagcaaattgaaaaatccaggctgtattataaacatcaattaaaagaagatctgggcctgggccttttgatgtttcctgaccagcttggatgctgtcacctttgtctactcagaagggagtaaaggcagtctaaagtcatcctcgccatcctcgccttcctggtgtgattcgcagaagtagaaagaacaccagcagctgctttgaaataccctggagctggcagatgatggacaggaaacagacagctgagaccctgctgaccctgtctcctgctgaagttgccaacctcaaggaagggatcaatttttttcgaaataagactactgggaaagagtacattttatacaaggagaaggaccatctaaaggcatgcaagaacctctgcaagcaccagggaggcctgttcatgaaagacatcgaggatttagatggaaggtccgttaaatgcacaaagcacaactggaagttagacgtgagcaccatgaaatatatcaaccctccagggagcttctgtcaagacgagctcgttattgaaatggatgaaaacaatgggctttccctggtagaactgaaccctcctaacccctgggactctgatcccaggtctcctgaagaattagcttttggggaagtacagataacatatctcactcatgcctgcatggacctcaagttgggagacaagcgaatggtatttgacccttggttaattggccctgcttttgcccgaggatggtggttgctacatgagcctccatctgactggttggagaggctgtgcaaagcagacctcatttatatcagccacatgcactcagaccacctgagctaccctaccctgaagcagctttcccagagacgaccagacattcccatttatgttggcgacacagaaaggcctgtgttttggaacctggatcagagtggcgtcgggttaactaacatcaacgtggttccatttggaatatggcaacaggtagacaaaagtctgcggttcatgatcttgatggacggcgttcatcctgagatggacacatgcattatcgtggagtacaaaggtcataaaatactcaacacagtggactgcaccagacccaatgggggaaggcttcctgagaaagttgctctaatgatgagtgatttcgcaggaggtgcatcaggctttccaatgactttcagtggtggaaaatttactgaggaatggaaagcccagttcattaaggctgaaagaagaaagcttctgaattacaaagctcagctggtgaaggacctgcagccccgaatctactgtccgtttgctgggtactttgtggagtctcacccatctgacaagtacattaaggaaacaaacaccaaaaatgacccaaatcagctcaacaatcttatcaggaaaaactctgacgtggtgacatggaccccacgacctggcgctgtcctcgaccttggcaggatgctgaaggacccaacagacagcaagggcattgtggagcctccagaggggacaaagatttacaaggattcctgggactttggcccgtacctggagatcttgaattctgctgtcagagatgaaatcttctgtcattcatcctggattaaagagtacttcacgtgggctggatttaagaattacaacctggtggtcaggatgattgaaacagatgaagatttcagcccttttcctggagggtacgactatctggtggactttctagatttatcctttccgaaagaaagacccagccgggagcatccttatgaagaaatccatagccgggtggatgtcatcaggtacgtggtgaagaacggcctgctgtgggatgatctgtatattggattccagacccgattgctgcgggaccctgatatataccatcatctgttttggaatcattttcagataaaactccctctaacaccacccaactggaagtcgttcctaatgcactgtgattagtctggacctggtaagtcccaggaccccagcccagaggatggtgcctgaacattcaagatgggtctcccctgcttcgataaacctttctgga

SEQ ID NO: 15 - DNA, 1165 bp, Mus musculus
  CDS (8)..(1117)
aagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacg